(19) United States
(12) Patent Application Publication
(10) Pub. No.: US 2020/0308632 A1
(43) Pub. Date: Oct. 1, 2020

(54) Methods for Detecting Microbes

(71) Applicant: Monsanto Technology LLC, St. Louis, MO (US)

(72) Inventors: Adil Anjem, St. Louis, MO (US); Amy L. Caruano-Yzermans, St. Louis, MO (US); Todd A. Ciche, Chesterfield, MO (US); Julia L. Stevens, St. Louis, MO (US); Keith H. Turner, St. Louis, MO (US)

(21) Appl. No.: 16/642,339

(22) PCT Filed: Aug. 29, 2018
(86) PCT No.: PCT/US18/48566

§ 371 Date: Feb. 26, 2020

Related U.S. Application Data
(60) Provisional application No. 62/552,225, filed on Aug. 30, 2017.

(51) Int. Cl. C12Q 1/689; C12Q 1/18; A01N 63/27; A01N 63/23; A01N 63/36

(52) U.S. Cl. CPC C12Q 1/689; C12Q 1/18; A01N 63/36; A01N 63/23; A01N 63/27

(57) ABSTRACT

Methods useful in detecting viable microbes in an agricultural composition are provided. Certain embodiments relate to methods in which a sample of an agricultural composition is obtained, and an amount of at least one pre-rRNA from at least one microbe is detected. The detected pre-rRNA from an agricultural sample may be compared with the amount of pre-rRNA from a control sample to determine the presence of a viable microbe.

### REFERENCE TO RELATED APPLICATION

This application claims the benefit of U.S. Provisional Application No. 62/552,225, filed Aug. 30, 2017, which is herein incorporated by reference in its entirety.

### FIELD OF THE INVENTION

The present disclosure relates to methods for detecting microbes, more specifically to methods for detecting viable microbes in an agricultural composition.

### BACKGROUND

Many microbes, notably bacteria and fungi, positively affect plant yield, health, and growth. Applying microorganisms to seeds is an efficient mechanism for associating microbes with crop plants, or introducing microbes into soil where they colonize the plant's roots. Beneficial microbes may improve fertilization, modulate plant growth hormones, or provide biopesticide and/or biocontrol activity.

There are significant technical challenges in maintaining viable microbe populations on seed throughout commercial seed treatment processes and storage. Considerable effort goes into identifying seed formulations that balance the biological requirements of the seed against the requirements of the microbial inoculant, which may differ from that of the seed. Additional seed treatment components, such as chemical pesticides or other inoculants may be incompatible with the beneficial microbes. An investigator needs an effective technique for detecting viable microbes on seeds in the presence of additional seed treatment components to evaluate microbial seed treatment formulations.

Culture-based counting of colony-forming units is the conventional method to quantify microbes. In this technique, an investigator dilutes and plates a microbe sample on suitable growth media. Over time, each plated viable cell gives rise to a single colony or Colony Forming Unit (CFU). The researcher counts the CFUs and calculates the sample's original cell concentration.

However, the method has several drawbacks. Microorganisms are difficult to cultivate from complex plant and environmental samples. The visual appearance of a colony in a cell culture requires significant growth—up to three days, and when counting colonies, it is uncertain if the colony arose from one cell or a group of cells—thus under representing the microbe concentration. Most culture-based detection methods can be time-consuming, lack sensitivity, and/or inaccurate.

Thus, there is a need for methods of detecting viable microbes in samples comprising plant material that are sensitive, efficient, and work in the presence of additional seed treatment components.

### SUMMARY

In one aspect, methods are provided for detecting a viable microbe in an agricultural composition, the methods comprising the steps of: (a) obtaining a first sample of an agricultural composition; (b) obtaining a control sample of the agricultural composition; (c) nutritionally stimulating the first sample; (d) incubating the first sample; (e) detecting an amount of at least one pre-rRNA from at least one microbe in the first sample; (f) detecting an amount of at least one pre-rRNA from at least one microbe in the control sample; and (g) comparing the amount of the at least one pre-rRNA from the at least one microbe in the first sample to the amount of the at least one pre-rRNA from the at least one microbe in the control sample; a greater amount of detected pre-rRNA in the first sample than in the control sample indicates the presence of a viable microbe. In certain embodiments, the agricultural composition comprises an agriculturally acceptable carrier. In some embodiments, the agricultural composition comprises plant material. In further embodiments, the agricultural composition comprises soil. In yet further embodiments, the agricultural composition comprises more than one microbe. In certain embodiments, the agricultural composition comprises a microbial community. In some embodiments, the agricultural composition comprises bacteria, fungi, and/or archaea. In further embodiments, the agricultural composition comprises bacteria.

In some embodiments, the bacteria in the agricultural composition comprises bacterial aggregates. For example, the bacteria may be viable but nonculturable, and may be Gram-negative bacteria or Gram-positive bacteria. In further embodiments, the agricultural composition comprises fungi. In yet further embodiments, the viable microbe in the agricultural composition are in a mid-logarithmic growth phase or a stationary growth phase.

In some embodiments, detecting the amount of the at least one pre-rRNA from at least one microbe in the control sample and detecting the amount of the at least one pre-rRNA from at least one microbe in the first sample is determined via RT-qPCR. In further embodiments, the plant material is a seed. In yet further embodiments, the agricultural composition comprises a seed treatment component. In some embodiments, the seed treatment component comprises a microbial inoculant. In further embodiments, the seed treatment component comprises a pesticide. In yet further embodiments, the pesticide is selected from the group consisting of at least one or more biocides, fungicides, herbicides, insecticides, miticides, nematicides, and rodenticides. In still further embodiments, the pesticide is selected from the group consisting of tioxazafen, clothianidin, ipconazole, imidacloprid, prothiconazol, fluoxastrobin, metalaxyl, trifloxystrobin, metalaxyl, and combinations thereof In some embodiments, the seed treatment component comprises one or more agriculturally acceptable nutrients and/or fertilizers. In certain embodiments, the seed treatment component comprises one or more plant signal molecules. In further embodiments, the seed treatment component comprises one or more adherents, adhesives, binders, buffers, coating agents, colorants, dispersants, fillers, polymers, polysaccharides, surfactants, and/or wetting agents.

In another aspect, methods are provided for determining the viability of a microbial inoculant, the methods comprising: (a) inoculating an agricultural composition with a microbe inoculant; (b) after a period of time, obtaining a first sample and a control sample from the agricultural composition with the microbe inoculant; (c) nutritionally stimulating the first sample; (d) incubating the first sample; (e) detecting the amount of the at least one pre-rRNA from at least one microbe in the first sample; (f) detecting the amount of the at least one pre-rRNA from at least one microbe in the control sample; (g) comparing the amount of the at least one pre-rRNA from at least one microbe in the first sample to the amount of the at least one pre-rRNA from at least one microbe in the control sample; and (h) quantifying viability of the at least one microbe in the first sample based on comparing the amount of the at least one pre-rRNA from at least one microbe in the first sample to the amount of the at least one pre-rRNA from at least one microbe in the control sample. In some embodiments, the agricultural composition comprises an agriculturally acceptable carrier. In certain embodiments, the agricultural composition comprises plant material. In further embodiments, the first sample and control sample comprise more than one microbe. In yet further embodiments, the first sample and control sample comprise a microbial community.

In certain embodiments, the at least one microbe is selected from the group consisting of bacteria, fungi, and archaea. In some embodiments, the at least one microbe comprises bacteria or bacterial aggregates. In further embodiments, the bacteria are viable but nonculturable. In yet further embodiments, the bacteria comprises Gram-negative bacteria. In still further embodiments, the bacteria comprises Gram-positive bacteria. In some embodiments, the at least one microbe comprises fungi. In further embodiments, the period of time is at least 3 days, at least one year, at least two years, or at least three years.

In some embodiments, the at least one microbe in the agricultural composition are in a mid-logarithmic growth phase. In certain embodiments, the at least one microbe in the agricultural composition are in a stationary growth phase. In further embodiments, detecting the amount of the at least one pre-rRNA from at least one microbe in the control sample and first sample is determined via RT-qPCR.

In yet further embodiments, the plant material comprises a seed. In still further embodiments, the agricultural composition comprises a seed treatment component. In some embodiments, the seed treatment component comprises a pesticide, for example a pesticide selected from the group consisting of at least one or more biocides, fungicides, herbicides, insecticides, miticides, nematicides, and rodenticides. In further embodiments, the pesticide is selected from the group consisting of tioxazafen, clothianidin, ipconazole, imidacloprid, prothiconazol, fluoxastrobin, metalaxyl, trifloxystrobin, metalaxyl, and combinations thereof.

In some embodiments, the seed treatment component comprises one or more agriculturally acceptable nutrients and/or fertilizers. In certain embodiments, the seed treatment component comprises one or more plant signal molecules. In further embodiments, the seed treatment component comprises one or more adherents, adhesives, binders, buffers, coating agents, colorants, dispersants, fillers, polymers, polysaccharides, surfactants, and/or wetting agents.

### BRIEF DESCRIPTION OF THE DRAWINGS

FIG. 1 shows a schematic of the 16S pre and mature rRNA gene for Pseudomonas entomophila and Bacillus thuringienis, the mature and ITS regions of the fungal species Penicillium restrictum, and the RT-qPCR assays used for detection in supporting figures;

FIG. 2 shows a linear response observed for RT-qPCR assays based on dilution series of total RNA measured by nanodrop;

FIG. 3 shows the relative expression of pre-rRNA in samples grown to the exponential verses the stationary phase for Pseudomonas entomophila and Bacillus thuringienis, respectively from the same total RNA amount measured by nanodrop;

FIG. 4 shows the relative expression of pre-rRNA in seed-treated with Pseudomonas entomophila in the presence and absence of seed treatment components from the same total RNA amount measured by nanodrop;

FIG. 5 shows the relative expression of pre-rRNA in seed-treated with Bacillus thuringiensis in the presence and absence of seed treatment component from the same total RNA amount measured by nanodrop;

FIG. 6 shows the relative expression of both mature and pre-rRNA in the fungal species Penicillium restrictum from 1 ml of suspended non-stimulated cells;

FIG. 7 shows the expression of pre-rRNA relative to rRNA along a time course in samples stimulated or unstimulated with Penicillium bilaii; and

FIG. 8 shows the expression of pre-rRNA in mixed microbial samples for P. entomophila and B. thuringiensis relative to rRNA amounts.

### DETAILED DESCRIPTION

### Abbreviations and Definitions

To facilitate understanding of the disclosure, several terms and abbreviations as used herein are defined as follows:

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. for example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein, is intended to qualify the numerical values that it modifies, denoting such a value as variable within a margin of error. When no margin of error, such as a standard deviation to a mean value given in a chart or table of data, is recited, the term “about” should be understood to mean that range which would encompass the recited value and the range which would be included by rounding up or down to that figure as well, considering significant figures.

The term “control sample,” as used herein, refers to any non-nutritionally stimulated sample used to establish a base or normal level of pre-rRNA. As opposed to a nutritionally stimulated sample of an agricultural composition (first sample), a control sample receives no stimulation. The non-stimulated sample or control sample serves as a baseline for quantifying the level of pre-rRNA produced in the nutritionally stimulated sample or first sample.

The term “detect” or “detection,” as used herein, refers to the determination of the existence, presence or fact of a target or signal in a limited portion of space, including but not limited to a sample or mixture. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified.

The term “incubating,” as used herein, refers to holding an item or sample (e.g., an inoculated culture, an enzymatic reaction mixture) at a temperature for a period or until a result (e.g., organism growth or reaction result) occurs.

The term “Internal Transcribed Spacer” or “ITS” as used herein, refers to the region located between the 18S and the 28S rRNA genes in the rRNA operon of fungal nuclear DNA. The ITS region is subdivided in the ITS-1 region, which separates the 18S and 5.8S rRNA genes, and the ITS-2 region which is found between the 5.8S and 28S rRNA genes.

The term “microbe” or “microorganism” refers to a unicellular or multi-cellular microscopic or macroscopic life form. Microorganisms include, but are not limited to, bacteria, viruses, fungi, algae, yeasts, protozoa, spirochetes, single-celled and multi-celled organisms that are either prokaryotes or eukaryotes that are known to those skilled in the art. Microbes include, but are not limited to, inoculated microbes or microbes present in the environment.

The term “nutritionally stimulating,” as used herein, refers to the cultivation of microorganisms in a nutrient media, including for example, water, nutrient chemicals, dissolved gases, and other media that provides the optimal environmental conditions for microbes continued growth and/or replication.

The term “plant material,” as used herein, refers to any part or parts of a plant taken individually or in a group. Examples include, but are not limited to, leaves, flowers, roots, seeds, stems, rhizomes, tubers, and other parts of a plant.

The term “RT-qPCR” is reverse transcription quantitative polymerase chain reaction referring to real-time PCR amplification of RNA into complementary DNA (cDNA) that is complementary to the RNA by using a reverse transcriptase.

The term “viable microorganism,” as used herein, refers to a microorganism that is capable of synthesizing precursor rRNA in response to nutritional stimulation.

The terms “precursor rRNA,” “precursor ribosomal RNA,” “pre-rRNA,” “preribosomal RNA,” or “pre-ribosomal RNA,” as used herein, refers to intermediates in rRNA synthesis generated by rapid nucleolytic cleavage of the polycistronic rrs-rrl-rrf operon transcript.

### Method for Detecting Viable Microbes in an Agricultural Composition

The disclosure provides a method for detecting viable microbes in an agricultural composition, the method comprising: obtaining a sample of an agricultural composition; nutritionally stimulating a first sample; incubating the first sample; detecting the amount of the at least one pre-rRNA from at least one microbe in the first sample; and comparing the amount of the at least one pre-rRNA from at least one microbe in the nutritionally stimulated first sample to the amount of the at least one pre-rRNA from at least one microbe in a control sample, thereby detecting viable microbes.

### Method for Determining the Viability of a Microbial Inoculant

The disclosure provides a method for determining the viability of a microbial inoculant, the method comprising: providing a microbial inoculant to an agricultural composition; after a period of time, obtaining a first sample comprising the inoculated agricultural composition; nutritionally stimulating the first sample; incubating the first sample; detecting the amount of the at least one pre-rRNA from at least one microbe in the first sample; comparing the amount of the at least one pre-rRNA from at least one microbe in the nutritionally stimulated sample to the amount of the at least one pre-rRNA from at least one microbe in a control sample, thereby detecting viable microbes; and determining the viability of the microbial inoculant based on the quantified viable microbes.

In each method, a microbial sample is nutritionally stimulated to induce viable cells to produce species-specific ribosomal RNA precursors (pre-rRNA). For example, a microorganism is placed in a defined medium composed of water, nutrient chemicals and/or dissolved gases, and allowed to grow (or multiply) to a desired culture density. The medium in some embodiments may contain chemicals that the cells utilize for their life processes to provide environmental conditions for their continued growth and/or replication. Non-viable cells produce no pre-rRNA. The pre-rRNA's in the stimulated aliquot are quantified relative to a non-stimulated control. Values of pre-rRNA that exceed those seen in non-stimulated control samples indicate the presence of viable cells.

### Agricultural Compositions

The methods described herein comprise an agricultural composition which comprises at least one microbe to be detected. In one embodiment, the agricultural composition comprises at least one microbe to be detected and an agriculturally acceptable carrier. In one embodiment, the agricultural composition comprises at least one microbe to be detected and plant material. In one embodiment, the agricultural composition comprises at least one microbe to be detected and soil. In one embodiment, the agricultural composition comprises at least one microbe to be detected and a seed treatment component.

### Agriculturally Acceptable Carriers

In one embodiment, the method includes detecting a microbe in an agricultural composition which comprises an agriculturally acceptable carrier. The agriculturally acceptable carrier in some embodiments may be used and/or combined with a microbial strain or isolate to improve the delivery or effectiveness of the microbial to a plant, plant part, or plant seed. In some embodiments, the agriculturally acceptable carrier may include a soil-compatible carrier, a seed-compatible carrier, and/or a foliar-compatible carrier. As used herein, the term “soil-compatible carrier” refers to a material that can be added or applied to a soil without causing/having an unduly adverse effect on plant yield, soil structure, soil drainage, or the like. The term “seed-compatible carrier” refers to a material that can be added or applied to a seed without causing/having an unduly adverse effect on the seed, seed germination, the plant that grows from the seed, or the like. The term “foliar-compatible carrier” refers to a material that can be added or applied to an above ground portion of a plant or plant part without causing/having an unduly adverse effect on plant yield, plant health, or the like. Selection of appropriate carrier materials will depend on the intended application(s) and the microorganism(s) present in the composition. The carrier material(s) may be selected and/or combined to provide a composition or formulation in the form of a liquid, gel, slurry, or solid. Compositions in some embodiments may comprise one or more liquid and/or gel carriers, and/or one or more aqueous and/or non-aqueous solvents. As used herein, the term “non-aqueous” may refer to a composition, solvent or substance that comprises no more than a trace amount of water (e.g., no more than 0.5% water by weight).

### Solid Carriers

According to some embodiments, the method includes detecting a microbe in an agricultural composition which comprises an agriculturally acceptable carrier, wherein the agriculturally acceptable carrier is in solid, powder form, and/or granular form. In other embodiments, the agricultural composition comprises one or more solid carriers. For example, in some embodiments, the agricultural compositions comprise one or more powders (e.g., wettable powders) and/or granules. Non-limiting examples of solid carriers that can be useful in agricultural compositions include peat-based powders and granules, freeze-dried powders, spray-dried powders, and combinations thereof.

### Liquid and Gel Carriers

In some embodiments, methods include obtaining a sample of an agricultural composition that is in liquid or gel form and/or comprise one or more liquid and/or gel carriers. In some embodiments, methods include obtaining a sample of an agricultural composition which may comprise a growth medium suitable for culturing one or more of the microorganisms in the composition. For example, agricultural compositions used in the methods herein may comprise a Czapek-Dox medium, a glycerol yeast extract, a mannitol yeast extract, a potato dextrose broth, and/or a YEM media. Commercial carriers may be used in accordance with a manufacturer's recommended amounts or concentrations.

### Plant Material

In some embodiments, methods include obtaining a sample of an agricultural composition which comprises at least one microbe to be detected and plant material. In some embodiment, plant material comprises a seed. The term “seed” as used herein, is not limited to any particular type of seed and can refer to seed from a single plant species, a mixture of seed from multiple plant species, a seed blend from various strains within a plant species, or a genetically modified seed. In some certain embodiments, the seed is a monocot. In other embodiments, the seed is corn. In some embodiments, the seed is wheat. In some certain embodiments, the seed is a dicot. In particular embodiments, the seed is soy.

In some embodiments, the seed is obtained from a package comprising seeds. The package may include any suitable packages. Examples of such suitable packages include, but are not limited to, bags, boxes, jugs, and single packages.

### Soil

In one embodiment, the method comprises obtaining an agricultural composition that comprises at least one microbe and soil. The soil may be any material that physically supports the root system of a plant and/or provides nutrients to the root system. The soil may be organic and/or synthetic soil.

Seed Treatment Components

In one embodiment, the method comprises obtaining an agricultural composition that comprises at least one microbe and a seed treatment component. Seed treatment components protect the seed and microbe during storage and after planting. Seed treatment components used in the methods herein are chosen from microbes, pesticides, nutrients and/or fertilizers, biostimulants, plant signal molecules, and combinations thereof. Seed treatment components may also include one or more adherents, adhesives, binders, buffers, coating agents, colorants, dispersants, fillers, polymers, polysaccharides, surfactants, and/or wetting agents.

In one embodiment, the method comprises obtaining an agricultural composition that, comprises a seed treatment component which comprises a seed treatment active, such as a biological agent and/or agrochemical. In other embodiments, the methods comprise obtaining an agricultural composition that comprises a seed treatment component which is a seed-finishing agent suitable for enhancing one or more physical properties of the exterior surfaces of the seeds. Seed treatment components may be applied in a dry state or a wet state (e.g., slurry).

### Seed Treatment Active

In the methods described herein, a seed treatment component may comprise a seed treatment active comprising one or more biological agents and/or agrochemicals. Seed treatment active is defined herein to include both seed treatment material and seed applied material. After being contacted by the seed treatment active (seed applied material or seed treated material), for purposes herein, the seeds are referred to as “treated seeds.”

In some embodiments, the seed treatment active comprises one or more pesticidal agents. Pesticidal agents include chemical pesticides and biopesticides or biocontrol agents. Various types of chemical pesticides and biopesticides include acaricides, insecticides, nematicides, fungicides, gastropodicides, herbicides, virucides, bactericides, and combinations thereof. Biopesticides or biocontrol agents may include bacteria, fungi, beneficial nematodes, and viruses that exhibit pesticidal activity.

### Acaricides, Insecticides and/or Nematicides

In some embodiments, the seed treatment active comprises one or more chemical acaricides, insecticides, and/or nematicides. Non-limiting examples of chemical acaricides, insecticides, and/or nematicides may include one or more carbamates, diamides, macrocyclic lactones, neonicotinoids, organophosphates, phenylpyrazoles, pyrethrins, spinosyns, synthetic pyrethroids, tetronic acids and/or tetramic acids. Non-limiting examples of chemical acaricides, insecticides and nematicides that can be useful in agricultural compositions include abamectin, acrinathrin, aldicarb, aldoxycarb, alpha-cypermethrin, betacyfluthrin, bifenthrin, cyhalothrin, cypermethrin, deltamethrin, esfenvalerate, etofenprox, fenpropathrin, fenvalerate, flucythrinate, fosthiazate, lambda-cyhalothrin, gamma-cyhalothrin, permethrin, tau-fluvalinate, transfluthrin, zeta-cypermethrin, cyfluthrin, bifenthrin, tefluthrin, eflusilanat, fubfenprox, pyrethrin, resmethrin, imidacloprid, acetamiprid, thiamethoxam, nitenpyram, thiacloprid, dinotefuran, clothianidin, chlorfluazuron, diflubenzuron, lufenuron, teflubenzuron, triflumuron, novaluron, flufenoxuron, hexaflumuron, bistrifluoron, noviflumuron, buprofezin, cyromazine, methoxyfenozide, tebufenozide, halofenozide, chromafenozide, endosulfan, fipronil, ethiprole, pyrafluprole, pyriprole, flubendiamide, chlorantraniliprole (e.g., Rynaxypyr), cyazypyr, emamectin, emamectin benzoate, abamectin, ivermectin, milbemectin, lepimectin, tebufenpyrad, fenpyroximate, pyridaben, fenazaquin, pyrimidifen, tolfenpyrad, dicofol, cyenopyrafen, cyflumetofen, acequinocyl, fluacrypyrin, bifenazate, diafenthiuron, etoxazole, clofentezine, spinosad, triarathen, tetradifon, propargite, hexythiazox, bromopropylate, chinomethionat, amitraz, pyrifluquinazon, pymetrozine, flonicamid, pyriproxyfen, diofenolan, chlorfenapyr, metaflumizone, indoxacarb, chlorpyrifos, spirodiclofen, spiromesifen, spirotetramat, pyridalyl, spinctoram, acephate, triazophos, profenofos, oxamyl, spinetoram, fenamiphos, fenamipclothiahos, 4-{[(6-chloropyrid-3-yl)methyl](2,2-difluoroethyl)amino}furan-2(5H)-one, 3,5-disubstituted-1,2,4-oxadiazole compounds, 3-phenyl-5-(thien-2-yl)-1,2,4-oxadiazole, cadusaphos, carbaryl, carbofuran, ethoprophos, thiodicarb, aldicarb, aldoxycarb, metamidophos, methiocarb, sulfoxaflor, methamidophos, cyantraniliprole and tioxazofen and combinations thereof. Additional non-limiting examples of chemical acaricides, insecticides, and/or nematicides may include one or more of abamectin, aldicarb, aldoxycarb, bifenthrin, carbofuran, chlorantraniliporle, chlothianidin, cyfluthrin, cyhalothrin, cypermethrin, cyantraniliprole, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, spinetoram, spinosad, spirodichlofen, spirotetramat, tefluthrin, thiacloprid, thiamethoxam, tioxazofen and/or thiodicarb, and combinations thereof.

Additional non-limiting examples of acaricides, insecticides and nematicides that may be included or used in compositions in some embodiments may be found in Steffey and Gray, Managing Insect Pests, Illinois Agronomy Handbook (2008); and Niblack, Nematodes, Illinois Agronomy Handbook (2008), the contents and disclosures of which are incorporated herein by reference. Non-limiting examples of commercial insecticides which may be suitable for the compositions disclosed herein include CRUISER (Syngenta, Wilmington, Delware), GAUCHO and PONCHO (Gustafson, Plano, Tex.). Active ingredients in these and other commercial insecticides may include thiamethoxam, clothianidin, and imidacloprid. Commercial acaricides, insecticides, and/or nematicides may be used in accordance with a manufacturer's recommended amounts or concentrations.

In some embodiments, the seed treatment active comprises one or more biopesticidal agents the presence and/or output of which is toxic to an acarid, insect and/or nematode. For example, the seed treatment active may comprise one or more of Bacillus firmus I-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43, and/or Metarhizium anisopliae BIO-1020, TAE-001; deposited as DSM 3884, DSM 3885, ATCC 90448, SD 170 and ARSEF 7711), Paecilomyces fumosoroseus FE991, and combinations thereof.

### Fungicides

In some embodiments, the seed treatment active comprises one or more chemical fungicides. Non-limiting examples of chemical fungicides may include one or more aromatic hydrocarbons, benzthiadiazole, carboxylic acid amides, morpholines, phenylamides, phosphonates, thiazolidines, thiophene, quinone outside inhibitors and strobilurins, such as azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester, and 2-(2-(3-(2,6-dichlorophenyl)-1-methyl-allylideneaminooxymethyl)-phenyl)-2-methoxyimino-N-methyl-acetamide, carboxamides, such as carboxanilides (e.g., benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyra-zole-4-carboxamide, N-(2-(1,3,3-trimethylbutyl)-phenyl)-1,3-dimethyl-5-fluoro-1H-pyrazole-4-carboxamide), carboxylic morpholides (e.g., dimethomorph, flumorph, pyrimorph), benzoic acid amides (e.g., flumetover, fluopicolide, fluopyram, zoxamide), carpropamid, dicyclomet, mandiproamid, fenehexamid, oxytetracyclin, silthiofam, and N-(6-methoxy-pyridin-3-yl) cyclopropanecarboxylic acid amide, spiroxamine, azoles, such as triazoles (e.g., azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole) and imidazoles (e.g., cyazofamid, imazalil, pefurazoate, prochloraz, triflumizol); heterocyclic compounds, such as pyridines (e.g., fluazinam, pyrifenox (cf.D1b), 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine), pyrimidines (e.g., bupirimate, cyprodinil, diflumetorim, fenarimol, ferimzone, mepanipyrim, nitrapyrin, nuarimol, pyrimethanil), piperazines (e.g., triforine), pyrroles (e.g., fenpiclonil, fludioxonil), morpholines(e.g., aldimorph, dodemorph, dodemorph-acetate, fenpropimorph, tridemorph), piperidines (e.g., fenpropidin); dicarboximides (e.g., fluoroimid, iprodione, procymidone, vinclozolin), non-aromatic 5-membered heterocycles (e.g., famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester), acibenzolar-S-methyl, ametoctradin, amisulbrom, anilazin, blasticidin-S, captafol, captan, chinomethionat, dazomet, debacarb, diclomezine, difenzoquat, difenzoquat-methyl sulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole and 5-chloro-7-(4-methylpiperidin-1-yl)-6-(2,4,6-trifluorophenyl)[1,2,4]triazolo-[1,5-a]pyrimidine; benzimidazoles, such as carbendazim; and other active substances, such as guanidines (e.g., guanidine, dodine, dodine free base, guazatine, guazatine-acetate, iminoctadine), iminoctadine-triacetate and iminoctadine-tris(albesilate); antibiotics (e.g., kasugamycin, kasugamycin hydrochloride-hydrate, streptomycin, polyoxine and validamycin A), nitrophenyl derivates (e.g., binapacryl, dicloran, dinobuton, dinocap, nitrothal-isopropyl, tecnazen); organometal compounds (e.g., fentin salts, such as fentin-acetate, fentin chloride, fentin hydroxide); sulfur-containing heterocyclyl compounds (e.g., dithianon, isoprothiolane), organophosphorus compounds (e.g., edifenphos, fosetyl, iprobenfos, phosphorus acid and its salts, pyrazophos, tolclofos-methyl), organochlorine compounds (e.g., chlorothalonil, dichlofluanid, dichlorophen, flusulfamide, hexachlorobenzene, pencycuron, pentachlorphenole and its salts, phthalide, quintozene, thiophanate-methyl, thiophanates, tolylfluanid, N-(4-chloro-2-nitro-phenyl)-N-ethyl-4-methyl-benzenesulfonamide) and inorganic active substances (e.g., Bordeaux mixture, copper acetate, copper hydroxide, copper oxychloride, basic copper sulfate, sulfur) and combinations thereof In an aspect, the seed treatment active comprises comprise acibenzolar-S-methyl, azoxystrobin, benalaxyl, bixafen, boscalid, carbendazim, cyproconazole, dimethomorph, epoxiconazole, fludioxonil, fluopyram, fluoxastrobin, flutianil, flutolanil, fluxapyroxad, fosetyl-A1, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thiabendazole, thifluzamide, thiophanate, tolclofos-methyl, trifloxystrobin and triticonazole, and combinations thereof.

For additional examples of fungicides that may be included in the seed treatment active compositions in some embodiments see, e.g., Bradley, Managing Diseases, Illinois Agronomy Handbook (2008), the content and disclosure of which are incorporated herein by reference. Fungicides useful for compositions in some embodiments may exhibit activity against one or more fungal plant pathogens, including but not limited to Phytophthora, Rhizoctonia, Fusarium, Pythium, Phomopsis, Selerotinia or Phakopsora, and combinations thereof. Non-limiting examples of commercial fungicides which may be suitable for the compositions in some embodiments include PROTÉGÉ, RIVAL or ALLEGIANCE FL or LS (Gustafson, Plano, Tex.), WARDEN RTA (Agrilance, St. Paul, Minn.), APRON XL, APRON MAXX RTA or RFC, MAXIM 4FS or XL (Syngenta, Wilmington, De.), CAPTAN (Arvesta, Guelph, Ontario) and PROTREAT (Nitragin Argentina, Buenos Ares, Argentina). Active ingredients in these and other commercial fungicides include, but are not limited to, fludioxonil, mefenoxam, azoxystrobin and metalaxyl. Commercial fungicides may be used in accordance with a manufacturer's recommended amounts or concentrations.

In some embodiments, the seed treatment active comprises one or more biopesticidal agents the presence and/or output of which is toxic to at least one fungus and/or bacteria. For example, the seed treatment active may comprise one or more of Ampelomyces quisqualis AQ 10® (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens FZB42, Bacillus amyloliquefaciens NRRL B-50349, Bacillus subtilis ATCC 55078, Bacillus subtilis ATCC 55079, Bacillus thuringiensis AQ52 (NRRL B-21619), Candida oleophila I-182 (e.g., ASPIRE® from Ecogen Inc., USA), Candida saitoana BIOCURE® (in mixture with lysozyme; BASF, USA) and BIOCOAT® (ArystaLife Science, Ltd., Cary, NC), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochium dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone Biolnnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANTSHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERNIA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma vixens GL-21 (SOILGARD®, Certis LLC, USA), and combinations thereof.

### Herbicides

In some embodiments, the seed treatment active comprises one or more suitable chemical herbicides. The herbicides may be a pre-emergent herbicide, a post-emergent herbicide, or a combination thereof. Non-limiting examples of chemical herbicides may comprise one or more acetyl CoA carboxylase (ACCase) inhibitors, acetolactate synthase (ALS) inhibitors, acetanilides, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvylshikimate-3-phosphate (EPSP) synthase inhibitors, glutamine synthetase inhibitors, dihydropteroate synthetase inhibitors, mitosis inhibitors, 4-hydroxyphenyl-pyruvate-dioxygenase (4-HPPD) inhibitors, synthetic auxins, auxin herbicide salts, auxin transport inhibitors, nucleic acid inhibitors and/or one or more salts, esters, racemic mixtures and/or resolved isomers thereof. Non-limiting examples of chemical herbicides that can be useful in agricultural compositions include 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluro, clethodim, clodinafop, clomazone, cyanazine, cycloxydim, cyhalofop, desmedipham, desmetryn, dicamba, diclofop, dimefuron, diflufenican, diuron, dithiopyr, ethofumesate, fenoxaprop, foramsulfuron, fluazifop, fluazifop-P, flufenacet, fluometuron, flufenpyr-ethyl, flumiclorac, flumiclorac-pentyl, flumioxazin, fluoroglycofen, fluthiacet-methyl, fomesafen, glyphosate, glufosinate, halosulfuron, haloxyfop, hexazinone, iodosulfuron, indaziflam, imazamox, imazaquin, imazethapyr, ioxynil, isoproturon, isoxaflutole, lactofen, linuron, mecoprop, mecoprop-P, mesosulfuron, mesotrion, metamitron, metazochlor, methibenzuron, metolachlor (and S-metolachlor), metoxuron, metribuzin, monolinuron, oxadiargyl, oxadiazon, oxaziclomefone, oxyfluorfen, phenmedipham, pretilachlor, profoxydim, prometon, prometrn, propachlor, propanil, propaquizafop, propisochlor, propoxycarbazone, pyraflufen-ethyl, pyrazon, pyrazolynate, pyrazoxyfen, pyridate, quizalofop, quizalofop-P (e.g., quizalofop-ethyl, quizalofop-P-ethyl, clodinafop-propargyl, cyhalofop-butyl, diclofop- methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins described in U.S. Pat. No.: 7,989,393), thiencarbazone-methyl, thenylchlor, tralkoxydim, triclopyr, trietazine, trifloxysulfuron, tropramezone, salts and esters thereof racemic mixtures and resolved isomers thereof and combinations thereof In an embodiment, seed treatment active compositions comprise acetochlor, clethodim, dicamba, flumioxazin, fomesafen, glyphosate, glufosinate, mesotrione, quizalofop, saflufenacil, sulcotrione, S-3100 and/or 2,4-D, and combinations thereof.

Additional examples of herbicides that may be included in compositions in some embodiments may be found in Hager, Weed Management, Illinois Agronomy Handbook (2008); and Loux et al., Weed Control Guide for Ohio, Indiana and Illinois (2015), the contents and disclosures of which are incorporated herein by reference. Commercial herbicides may be used in accordance with a manufacturer's recommended amounts or concentrations.

In some embodiments, the seed treatment active comprises one or more biopesticidal agents the presence and/or output of which is toxic to at least one plant, including for example, weeds. Examples of biopesticides that may be included or used in compositions in some embodiments may be found in Burges, supra; Hall & Menn, Biopesticides: Use and Delivery (Humana Press) (1998); McCoy et al., Entomogenous fungi, in CRC Handbook of Natural Pesticides. Microbial Pesticides, Part A. Entomogenous Protozoa and Fungi (C. M. Inoffo, ed.), Vol. 5: 151-236 (1988); Samson et al., Atlas of Entomopathogenic Fungi (Springer-Verlag, Berlin) (1988); and deFaria and Wraight, Mycoinsecticides and Mycoacaricides: A comprehensive list with worldwide coverage and international classification of formulation types, Biol. Control (2007), the contents and disclosures of which are incorporated herein by reference.

### Additional Agents

In some embodiments, the seed treatment active comprises one or more additional agent.

In some embodiments, the seed treatment active comprises one or more beneficial biostimulants and/or microbial inoculants. Biostimulants or inoculants may enhance ion uptake, nutrient uptake, nutrient availability or delivery, or a combination thereof. Non-limiting examples of biostimulants or inoculants that may be included or used in compositions may include bacterial extracts (e.g., extracts of one or more diazotrophs, phosphate-solubilizing microorganisms and/or biopesticides), fungal extracts, humic acids (e.g., potassium humate), fulvic acids, myo-inositol, and/or glycine, and any combinations thereof. According to some embodiments, the biostimulants or inoculants may comprise one or more Azospirillum (e.g., an extract of media comprising A. brasilense INTA Az-39), one or more Bradyrhizobium (e.g., an extract of media comprising B. elkanii SEMIA 501, B. elkanii SEMIA 587, B. elkanii SEMIA 5019, B. japonicum NRRL B-50586 (also deposited as NRRL B-59565), B. japonicum NRRL B-50587 (also deposited as NRRL B-59566), Bacillus amyloliquefaciens TJ1000 (also known as 1BE, isolate ATCC BAA-390), B. japonicum NRRL B-50588 (also deposited as NRRL B-59567), B. japonicum NRRL B-50589 (also deposited as NRRL B-59568), B. japonicum NRRL B-50590 (also deposited as NRRL B-59569), B. japonicum NRRL B-50591 (also deposited as NRRL B-59570), Trichoderma virens G1-3 (ATCC 57678), Trichoderma virens G1-21 (Thermo Trilogy Corporation, Wasco, Calif.), Trichoderma virens G1-3 and Bacillus amyloliquefaciens FZB24, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma virens G1-21 and Bacillus amyloliquefaciens NRRL B-50349, Trichoderma virens G1-21 and Bacillus amyloliquefaciens TJ1000, Trichoderma viride TRIECO® (Ecosense Labs (India) Pvt. Ltd., India, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ), B. japonicum NRRL B-50592 (also deposited as NRRL B-59571), B. japonicum NRRL B-50593 (also deposited as NRRL B-59572), B. japonicum NRRL B-50594 (also deposited as NRRL B-50493), B. japonicum NRRL B-50608, B. japonicum NRRL B-50609, B. japonicum NRRL B-50610, B. japonicum NRRL B-50611, B. japonicum NRRL B-50612, B. japonicum NRRL B-50726, B. japonicum NRRL B-50727, B. japonicum NRRL B-50728, B. japonicum NRRL B-50729, B. japonicum NRRL B-50730, B. japonicum SEMIA 566, B. japonicum SEMIA 5079, B. japonicum SEMIA 5080, B. japonicum USDA 6, B. japonicum USDA 110, B. japonicum USDA 122, B. japonicum USDA 123, B. japonicum USDA 127, B. japonicum USDA 129 and/or B. japonicum USDA 532C), one or more Rhizobium extracts (e.g., an extract of media comprising R. leguminosarum S012A-2), one or more SinoRhizobium extracts (e.g., an extract of media comprising S. fredii CCBAU114 and/or S. fredii USDA 205), one or more Penicillium extracts (e.g., an extract of media comprising P. bilaiae ATCC 18309, P. bilaiae ATCC 20851, P. bilaiae ATCC 22348, P. bilaiae NRRL 50162, P. bilaiae NRRL 50169, P. bilaiae NRRL 50776, P. bilaiae NRRL 50777, P. bilaiae NRRL 50778, P. bilaiae NRRL 50777, P. bilaiae NRRL 50778, P. bilaiae NRRL 50779, P. bilaiae NRRL 50780, P. bilaiae NRRL 50781, P. bilaiae NRRL 50782, P. bilaiae NRRL 50783, P. bilaiae NRRL 50784, P. bilaiae NRRL 50785, P. bilaiae NRRL 50786, P. bilaiae NRRL 50787, P. bilaiae NRRL 50788, P. bilaiae RS7B-SD1, P. brevicompactum AgRF18, P. canescens ATCC 10419, P. expansum ATCC 24692, P. expansum YT02, P. fellatanum ATCC 48694, P. gaestrivorus NRRL 50170, P. glabrum DAOM 239074, P. glabrum CBS 229.28, P. janthinellum ATCC 10455, P. lanosocoeruleum ATCC 48919, P. radicum ATCC 201836, P. radicum FRR 4717, P. radicum FRR 4719, P. radicum N93/47267 and/or P. raistrickii ATCC 10490), one or more Pseudomonas extracts (e.g., an extract of media comprising P. jessenii PS06), one or more acaricidal, insecticidal and/or nematicidal extracts (e.g., an extract of media comprising Bacillus firmus I-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43 and Metarhizium anisopliae BIO-1020, TAE-001; deposited as DSM 3884, DSM 3885, ATCC 90448, SD 170 and ARSEF 7711) and/or Paecilomyces fumosoroseus FE991), and/or one or more fungicidal extracts (e.g., an extract of media comprising Ampelomyces quisqualis AQ 10® (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens NRRL B-50349, Bacillus amyloliquefaciens TJ1000 (also known as 1BE, isolate ATCC BAA-390), Bacillus thuringiensis AQ52 (NRRL B-21619), Candida oleophila I-82 (e.g., ASPIRE® from Ecogen Inc., USA), Candida saitoana BIOCURE® (in mixture with lysozyme; BASF, USA) and BIOCOAT® (ArystaLife Science, Ltd., Cary, NC), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochium dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone Biolnnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANTSHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERMA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma virens GL-21 (SOILGARD®, Certis LLC, USA), Trichoderma virens G1-3, ATCC 57678, Trichoderma virens G1-21 (Thermo Trilogy Corporation, Wasco, Calif.), Trichoderma virens G1-3 and Bacillus amyloliquefaciens FZB2, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma virens G1-21 and Bacillus amyloliquefaciens NRRL B-50349, Trichoderma virens G1-21 and Bacillus amyloliquefaciens TJ1000, Trichoderma viride TRIECO® (Ecosense Labs. (India) Pvt. Ltd., Indien, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ)), and combinations thereof.

In some embodiments, the seed treatment active comprises one or more beneficial microbes. Non-limiting examples of such microbes include beneficial microbes selected from the following genera: Actinomycetes, Agrobacterium, Arthrobacter, Alcaligenes, Acinetobacter spp, Azospirillum spp, Aureobacterium, Azobacter, Azorhizobium, Bacillus, Beijerinckia, Bradyrhizobium, Brevibacillus, Burkholderia, Chromobacterium, Chryseomonas spp., Clostridium, Clavibacter, Comamonas, Corynebacterium, Curtobacterium, Enterobacter, EuPenicillium spp., Exiguobacterium spp., Flavobacterium, Gluconobacter, Hydrogenophaga, Hymenoscyphous, Klebsiella, Kluyvera spp., Methylobacterium, Paenibacillus, Pasteuria, Photorhabdus, Phyllobacterium, Pseudomonas, Rhizobium, Rhizobacter, Rhizopogon, Serratia, Sinorhizobium, Sphingobacterium, Swaminathania spp., Stenotrophomonas, Streptomyces spp., Thiobacillus, Variovorax, Vibrio, Xanthobacter, Xanthomonas and Xenorhabdus, or any combination thereof. According to some embodiments, the seed treatment active comprises one or more of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium subtsugae, Pasteuria penetrans, Pasteuria usage, and Pseudomona fluorescens. According to some embodiments, a microbe may comprise a fungus of the genus Alternaria, Ampelomyces, Arthrobotrys spp., Aspergillus, Aureobasidium, Beauveria, Candida spp., Colletotrichum, Coniothyrium, Gigaspora spp., Gliocladium, Glomus spp., Laccaria spp., Metarhizium, Mucor spp., Muscodor, Oidiodendron spp., Paecilomyces, Penicillium spp., Pisolithus spp., Scleroderma, Trichoderma, Typhula, Ulocladium, and Verticillium. In another aspect, a fungus is Beauveria bassiana, Coniothyrium minitans, Gliocladium virens, Muscodor albus, Paecilomyces lilacinus, or Trichoderma polysporum.

In some embodiments, the seed treatment active comprises one or more lipo-chitooligosaccharides (LCOs), chitin oligomer(s) and/or chitosan oligomer(s) (collectively referred to hereinafter as COs), and/or chitinous compounds.

LCOs, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end. As understood in the art, LCOs differ in the number of GlcNAc residues in the backbone, in the length and degree of saturation of the fatty acyl chain and in the substitutions of reducing and non-reducing sugar residues. See, e.g., Denarie et al., Ann. Rev. Biochem. 65: 503 (1996); Diaz et al., Mol. Plant-Microbe Interactions 13: 268 (2000); Hungria et al., Soil Biol. Biochem. 29: 819 (1997); Hamel et al., Planta 232: 787 (2010); and Prome et al., Pure & Appl. Chem. 70(1): 55 (1998), the contents and disclosures of which are incorporated herein by reference.

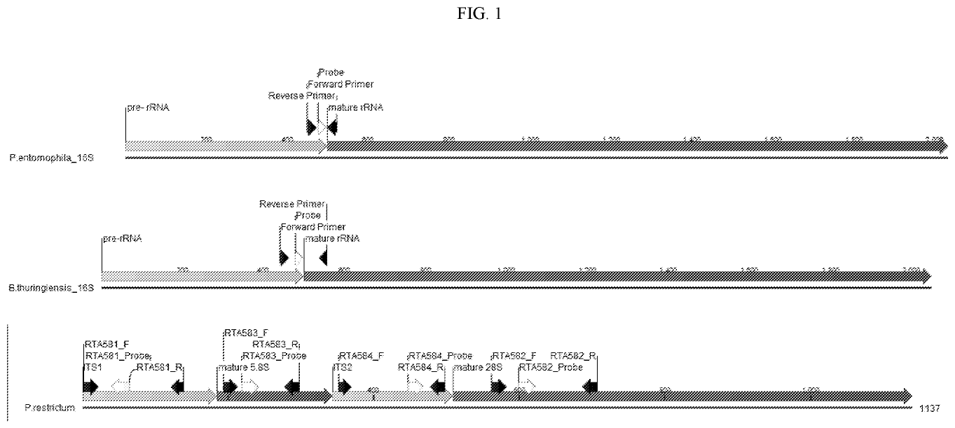

LCOs may be synthetic or obtained from any suitable source. See, e.g., WO 2005/063784, WO 2007/117500 and WO 2008/071674, the contents and disclosures of which are incorporated herein by reference. In some aspects, a synthetic LCO may have the basic structure of a naturally occurring LCO but contains one or more modifications or substitutions, such as those described in Spaink, Crit. Rev. Plant Sci. 54: 257 (2000). LCOs and precursors for the construction of LCOs (e.g., COs, which may themselves be useful as a biologically active ingredient) can be synthesized by genetically engineered organisms. See, e.g., Samain et al., Carbohydrate Res. 302: 35 (1997); Cottaz et al., Meth. Eng. 7(4): 311 (2005); and Samain et al., J. Biotechnol. 72: 33 (1999) (e.g., FIG. 1 therein, which shows structures of COs that can be made recombinantly in E. coli harboring different combinations of genes nodBCHL), the contents and disclosures of which are incorporated herein by reference.

LCOs (and derivatives thereof) may be included or utilized in compositions in various forms of purity and can be used alone or in the form of a culture of LCO-producing bacteria or fungi. For example, OPTIMIZE® (commercially available from Monsanto Company (St. Louis, Mo.)) contains a culture of Bradyrhizobium japonicum that produces LCO. Methods to provide substantially pure LCOs include removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC and the purified LCO molecules can be freeze-dried for long-term storage. According to some embodiments, the LCO(s) included in agricultural compositions is/are at least 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% pure. Compositions and methods in some embodiments may comprise analogues, derivatives, hydrates, isomers, salts and/or solvates of LCOs. LCOs may be incorporated into agricultural compositions in any suitable amount(s)/concentration(s). For example, agricultural compositions comprise about 1×10⁻²⁰ M to about 1×10⁻¹ M LCO(s). For example, agricultural compositions can comprise about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, 1×10⁻¹ M of one or more LCOs. In an aspect, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M. In an aspect, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M. The amount/concentration of LCO may be an amount effective to impart a positive trait or benefit to a plant, such as to enhance the disease resistance, growth and/or yield of the plant to which the composition is applied. According to some embodiments, the LCO amount/concentration is not effective to enhance the yield of the plant without beneficial contributions from one or more other constituents of the composition, such as CO and/or one or more pesticides.

In some embodiments, the seed treatment active comprises one or more chitin oligomers and/or chitosan oligomers. See, e.g., D3 Haeze et al., Glycobiol. 12(6): 79R (2002); Demont-Caulet et al., Plant Physiol. 120(1): 83 (1999); Hanel et al., Planta 232: 787 (2010); Muller et al., Plant Physiol. 124: 733 (2000); Robina et al., Tetrahedron 58: 521-530 (2002); Rouge et al., Docking of Chitin Oligomers and Nod Factors on Lectin Domains of the LysM-RLK Receptors in the Medicago-Rhizobium Symbiosis, in The Molecular Immunology of Complex Carbohydrates-3 (Springer Science, 2011); Van der Holst et al., Curr. Opin. Struc. Biol. 11: 608 (2001); and Wan et al., Plant Cell 21: 1053 (2009), the contents and disclosures of which are incorporated by reference. COs may be obtained from any suitable source. For example, COs may be derived from an LCO. For example, in an aspect, compositions comprise one or more COs derived from an LCO obtained (i.e., isolated and/or purified) from a strain of Azorhizobium, Bradyrhizobium (e.g., B. japonicum), Mesorhizobium, Rhizobium (e.g., R. leguminosarum), SinoRhizobium (e.g., S. meliloti), or mycorhizzal fungi (e.g., Glomus intraradicus). Alternatively, the CO may be synthetic. Methods for the preparation of recombinant COs are known in the art. See, e.g., Cottaz et al., Meth. Eng. 7(4): 311 (2005); Samain et al., Carbohydrate Res. 302: 35 (1997); and Samain et al., J. Biotechnol. 72: 33 (1999), the contents and disclosures of which are incorporated herein by reference.

COs (and derivatives thereof) may be included or utilized in compositions in various forms of purity and can be used alone or in the form of a culture of CO-producing bacteria or fungi. According to some embodiments, the CO(s) included in compositions may be at least 0.1%, 0.5%, 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, 99.5% or more pure. It is to be understood that agricultural compositions and methods herein can comprise hydrates, isomers, salts and/or solvates of COs. COs in some embodiments may be incorporated into compositions in any suitable amount(s)/concentration(s). For example, compositions in some embodiments may comprise about 1×10⁻²⁰ M to about 1×10⁻¹ M COs, such as about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, or 1×10⁻¹ M of one or more COs. For example, the CO concentration may be 1×10⁻¹⁴ M to 1×10⁻⁵ M, 1×10⁻¹² M to 1×10⁻⁶ M, or 1×10⁻¹⁰ M to 1×10⁻⁷ M. The amount/concentration of CO may be an amount effective to impart or confer a positive trait or benefit to a plant, such as to enhance the soil microbial environment, nutrient uptake, or increase the growth and/or yield of the plant to which the composition is applied. Compositions in some embodiments may comprise one or more suitable chitinous compounds, such as, for example, chitin (IUPAC: N-[5-[[3-acetylamino-4,5-dihydroxy-6-(hydroxymethypoxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol), and isomers, salts and solvates thereof.

Chitins and chitosans, which are major components of the cell walls of fungi and the exoskeletons of insects and crustaceans, are composed of GlcNAc residues. Chitins and chitosans may be obtained commercially or prepared from insects, crustacean shells, or fungal cell walls. Methods for the preparation of chitin and chitosan are known in the art. See, e.g., U.S. Pat. No. 4,536,207 (preparation from crustacean shells) and U.S. Pat. No. 5,965,545 (preparation from crab shells and hydrolysis of commercial chitosan); and Pochanavanich et al., Lett. Appl. Microbiol. 35: 17 (2002) (preparation from fungal cell walls).

Deacetylated chitins and chitosans may be obtained that range from less than 35% to greater than 90% deacetylation and cover a broad spectrum of molecular weights, e.g., low molecular weight chitosan oligomers of less than 15 kD and chitin oligomers of 0.5 to 2 kD; “practical grade” chitosan with a molecular weight of about 15 kD; and high molecular weight chitosan of up to 70 kD. Chitin and chitosan compositions formulated for seed treatment are commercially available. Commercial products include, for example, ELEXA® (Plant Defense Boosters, Inc.) and BEYOND™ (Agrihouse, Inc.).

In some embodiments, the seed treatment active comprises one or more suitable flavonoids, including, but not limited to, anthocyanidins, anthoxanthins, chalcones, coumarins, flavanones, flavanonols, flavans and isoflavonoids, as well as analogues, derivatives, hydrates, isomers, polymers, salts and solvates thereof. Flavonoids are phenolic compounds having the general structure of two aromatic rings connected by a three-carbon bridge. Classes of flavonoids are known in the art. See, e.g., Jain et al., J. Plant Biochem. & Biotechnol. 11: 1 (2002); and Shaw et al., Environ. Microbiol. 11:1867 (2006), the contents and disclosures of which are incorporated herein by reference. Several flavonoid compounds are commercially available. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, See, e.g. Ralston et al., Plant Physiol. 137: 1375 (2005).

In some embodiments, the seed treatment active comprises one or more flavanones, such as one or more of butin, eriodictyol, hesperetin, hesperidin, homoeriodictyol, isosakuranetin, naringenin, naringin, pinocembrin, poncirin, sakuranetin, sakuranin, and/or sterubin, one or more flavanonols, such as dihydrokaempferol and/or taxifolin, one or more flavans, such as one or more flavan-3-ols (e.g., catechin (C), catechin 3-gallate (Cg), epicatechins (EC), epigallocatechin (EGC) epicatechin 3-gallate (ECg), epigallcatechin 3-gallate (EGCg), epiafzelechin, fisetinidol, gallocatechin (GC), gallcatechin 3-gallate (GCg), guibourtinidol, mesquitol, robinetinidol, theaflavin-3-gallate, theaflavin-3′-gallate, theflavin-3,3′-digallate, thearubigin), flavan-4-ols (e.g., apiforol and/or luteoforol) and/or flavan-3,4-diols (e.g., leucocyanidin, leucodelphinidin, leucofisetinidin, leucomalvidin, luecopelargonidin, leucopeonidin, leucorobinetinidin, melacacidin and/or teracacidin) and/or dimers, trimers, oligomers and/or polymers thereof (e.g., one or more proanthocyanidins), one or more isoflavonoids, such as one or more isoflavones or flavonoid derivatives (e.g, biochanin A, daidzein, formononetin, genistein and/or glycitein), isoflavanes (e.g., equol, ionchocarpane and/or laxifloorane), isoflavandiols, isoflavenes (e.g., glabrene, haginin D and/or 2-methoxyjudaicin), coumestans (e.g., coumestrol, plicadin and/or wedelolactone), pterocarpans, roetonoids, neoflavonoids (e.g, calophyllolide, coutareagenin, dalbergichromene, dalbergin, nivetin), and/or pterocarpans (e.g., bitucarpin A, bitucarpin B, erybraedin A, erybraedin B, erythrabyssin II, erthyrabissin-1, erycristagallin, glycinol, glyceollidins, glyceollins, glycyrrhizol, maackiain, medicarpin, morisianine, orientanol, phaseolin, pisatin, striatine, trifolirhizin), and combinations thereof. Flavonoids and their derivatives may be included in compositions in any suitable form, including, but not limited to, polymorphic and crystalline forms. Flavonoids may be included in compositions in any suitable amount(s) or concentration(s). The amount/concentration of a flavonoid(s) may be an amount effective, which may be indirectly through activity on soil microorganisms or other means, such as to enhance plant nutrition and/or yield. According to some embodiments, a flavonoid amount/concentration may not be effective to enhance the nutrition or yield of the plant without the beneficial contributions from one or more other ingredients of the composition, such as LCO, CO, and/or one or more pesticides.

In some embodiments, the seed treatment active comprises one or more non-flavonoid nod-gene inducer(s), including, but not limited to, jasmonic acid ([1R-[1α,2β(Z)]]-3-oxo-2-(pentenyl)cyclopentaneacetic acid; JA), linoleic acid ((Z,Z)-9,12-Octadecadienoic acid) and/or linolenic acid ((Z,Z,Z)-9,12,15-octadecatrienoic acid), and analogues, derivatives, hydrates, isomers, polymers, salts and solvates thereof. Jasmonic acid and its methyl ester, methyl jasmonate (MeJA), collectively known as jasmonates, are octadecanoid-based compounds that occur naturally in some plants (e.g., wheat), fungi (e.g., Botryodiplodia theobromae, Gibbrella fujikuroi), yeast (e.g., Saccharomyces cerevisiae) and bacteria (e.g., Escherichia coli). Linoleic acid and linolenic acid may be produced during the biosynthesis of jasmonic acid.

Derivatives of jasmonic acid, linoleic acid, and linolenic acid that may be included or used in compositions in some embodiments include esters, amides, glycosides and salts thereof. Representative esters are compounds in which the carboxyl group of linoleic acid, linolenic acid, or jasmonic acid has been replaced with a —COR group, where R is an —OR¹ group, in which R¹ is: an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C2₋C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are each independently: a hydrogen; an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic addition, wherein the carboxylic acid is reacted with an alcohol in the presence of a catalytic amount of a mineral acid. Amides may also be prepared by known methods, such as by reacting the carboxylic acid with the appropriate amine in the presence of a coupling agent, such as dicyclohexyl carbodiimide (DCC), under neutral conditions. Suitable salts of linoleic acid, linolenic acid and jasmonic acid include, for example, base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing a solution of linoleic acid, linolenic acid, or jasmonic acid with a solution of the base. The salts may be precipitated from solution and collected by filtration, or may be recovered by other means such as by evaporation of the solvent.

In some embodiments, the seed treatment active comprises one or more plant growth regulators including, but not limited to, ethephon and/or thidiazuron.

In some embodiments, the seed treatment active comprises one or more karrakins, including but not limited to 2H-furo[2,3-c]pyran-2-ones, as well as analogues, derivatives, hydrates, isomers, polymers, salts and solvates thereof. Examples of biologically acceptable salts of karrakins include acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate and p-toluenesulphonic acid. Additional biologically acceptable metal salts may include alkali metal salts, with bases, examples of which include the sodium and potassium salts. Karrakins may be incorporated into compositions in any suitable amount(s) or concentration(s). For example, the amount/concentration of a karrakin may be an amount or concentration effective to impart or confer a positive trait or benefit to a plant, such as to enhance the disease resistance, growth and/or yield of the plant to which the composition is applied. In an aspect, a karrakin amount/concentration may not be effective to enhance the disease resistance, growth and/or yield of the plant without beneficial contributions from one or more other ingredients of the composition, such as a LCO, CO and/or one or more pesticides.

In some embodiments, the seed treatment active comprises one or more anthocyanidins and/or anthoxanthins, such as one or more of cyanidin, delphinidin, malvidin, pelargonidin, peonidin, petunidin, flavones (e.g., apigenin, baicalein, chrysin, 7,8-dihydroxyflavone, diosmin, flavoxate, 6-hydroxyflavone, luteolin, scutellarein, tangeritin and/or wogonin) and/or flavonols (e.g., amurensin, astragalin, azaleatin, azalein, fisetin, furanoflavonols galangin, gossypetin, 3-hydroxyflavone, hyperoside, icariin, isoquercetin, kaempferide, kaempferitrin, kaempferol, isorhamnetin, morin, myricetin, myricitrin, natsudaidain, pachypodol, pyranoflavonols quercetin, quericitin, rhamnazin, rhamnetin, robinin, rutin, spiraeoside, troxerutin and/or zanthorhamnin), and combinations thereof.

In some embodiments, the seed treatment active comprises one or more gluconolactone and/or an analogue, derivative, hydrate, isomer, polymer, salt and/or solvate thereof. Gluconolactone may be incorporated into compositions in any suitable amount(s)/concentration(s). For example, the amount/concentration of a gluconolactone amount/concentration may be an amount effective to impart or confer a positive trait or benefit to a plant, such as to enhance the disease resistance, growth and/or yield of the plant to which the composition is applied. In an aspect, the gluconolactone amount/concentration may not be effective to enhance the disease resistance, growth and/or yield of the plant without beneficial contributions from one or more other ingredients of the composition, such as a LCO, CO and/or one or more pesticides.

In some embodiments, the seed treatment active comprises one or more nutrient(s) and/or fertilizer(s), such as organic acids (e.g., acetic acid, citric acid, lactic acid, malic acid, taurine, etc.), macrominerals (e.g., phosphorous, calcium, magnesium, potassium, sodium, iron, etc.), trace minerals (e.g., boron, cobalt, chloride, chromium, copper, fluoride, iodine, iron, manganese, molybdenum, selenium, zinc, etc.), vitamins, (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K.), and/or carotenoids (α-carotene, β-carotene, cryptoxanthin, lutein, lycopene, zeaxanthin, etc.), and combinations thereof. In an aspect, agricultural compositions may comprise macro- and micronutrients of plants or microbes, including phosphorous, boron, chlorine, copper, iron, manganese, molybdenum and/or zinc. According to some embodiments, compositions may comprise one or more beneficial micronutrients. Non-limiting examples of micronutrients for use in compositions described herein may include vitamins, (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.), macrominerals (e.g., phosphorous, calcium, magnesium, potassium, sodium, iron, etc.), trace minerals (e.g., boron, cobalt, chloride, chromium, copper, fluoride, iodine, iron, manganese, molybdenum, selenium, zinc, etc.), organic acids (e.g., acetic acid, citric acid, lactic acid, malic acid, taurine, etc.), and combinations thereof. In a particular aspect, compositions may comprise phosphorous, boron, chlorine, copper, iron, manganese, molybdenum, and/or zinc, and combinations thereof. For compositions comprising phosphorous, it is envisioned that any suitable source of phosphorous may be used. For example, phosphorus may be derived from a rock phosphate source, such as monoammonium phosphate, diammonium phosphate, monocalcium phosphate, super phosphate, triple super phosphate, and/or ammonium polyphosphate, an organic phosphorous source, or a phosphorous source capable of solubilization by one or more microorganisms (e.g., Penicillium bilaiae).

### Additional Material

In some embodiments, the seed treatment component comprises one or more adherents, adhesives, binders, buffers, coating agents, colorants, dispersants, fillers, polymers, polysaccharides, surfactants, and/or wetting agents.

### Adherents

In some embodiments, the seed treatment component comprises one or more adherents. Examples of such materials include, but are not limited to, alginates; celluloses; such as hydroxymethyl celluloses, methyl celluloses, and hydroxymethyl propyl celluloses, dextrins, fats, gelatins, gum arabics, maltodextrins, molasses, oils, one or more mono- di- oligo- or polysaccharides, paraffinic hydrocarbon solvents, peptones, polyethylene glycol (PEG), polyvinyl acetate copolymers, polyvinyl acetates, polyvinyl alcohol copolymers, polyvinyl alcohols, polyvinyl pyrrolidones (PVP), proteins, proteins, starches, sugar alcohols, sugars, synthetic polymers, or syrups.

### Adhesives

In some embodiments, the seed treatment component comprises one or more adhesives. Examples of such materials include, but are not limited to, polyvinyl carboxymethylcellulose and natural and synthetic polymers in the form of powders, granules, or latexes, such as gum arabic, chitin, polyvinyl alcohol and polyvinyl acetate, as well as natural phospholipids, such as cephalins and lecithins, and synthetic phospholipids.

### Binders

In some embodiments, the seed treatment component comprises one or more binders. Examples of such materials include, but are not limited to, polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinyl acrylates; polyethylene oxide; acrylamide polymers and copolymers; polyhydroxyethyl acrylate, methylacrylamide monomers; and polychloroprene.

### Solvents

In some embodiments the seed treatment component may comprise one or more various solvents, such as organic, inorganic, non-aqueous and/or aqueous solvent(s). Examples of inorganic solvents include water, ammonia, and sulfur dioxide. Examples of commercially available organic solvents include pentadecane, ISOPAR M, ISOPAR V, and ISOPAR L (Exxon Mobil). Additional examples of solvents that may be included in compositions and formulations can be found in Burges, supra; Inoue & Horikoshi, J. Fermentation Bioeng. 71(3): 194 (1991), the contents and disclosures of which are incorporated herein by reference. According to some embodiments, an aqueous solvent, such as water, may be combined with a co-solvent, such as ethyl lactate, methyl soyate/ethyl lactate co-solvent blends (e.g., STEPOSOL, available from Stepan), isopropanol, acetone, 1,2-propanediol, n-alkylpyrrolidones (e.g., the AGSOLEX series, available from ISP), a petroleum based-oil (e.g., AROMATIC series and SOLVESSO series available from Exxon Mobil), isoparaffinic fluids (e.g., ISOPAR series, available from Exxon Mobil), cycloparaffinic fluids (e.g., NAPPAR 6, available from Exxon Mobil), mineral spirits (e.g., VARSOL series available from Exxon Mobil), and mineral oils (e.g., paraffin oil). According to some embodiments, compositions may comprise one or more co-solvent(s) in addition to an aqueous solvent or water. Such co-solvent(s) may include, for example, non-aqueous solvents, such as one or more the foregoing non-aqueous solvents.

### Buffers

In some embodiments, the seed treatment component comprises one or more buffers. The agriculturally acceptable buffers may be chosen to provide an aqueous suspension concentrate composition having a pH of less than 10, from about 5 to about 9, from about 6 to about 7. 5, and about 7.

### Stabilizers

The seed treatment component in some embodiments comprises one or more thickeners, rheology modifying agents, or stabilizing agents (“stabilizers”). Examples of stabilizers include anionic polysaccharides and cellulose derivatives. A stabilizer may comprise, for example, a clay, a silica, or a colloidal hydrophilic silica. Non-limiting examples of commercially available stabilizers include KELZAN CC (Kelco), methyl cellulose, carboxymethylcellulose and 2-hydroxyethylcellulose, hydroxymethylcellulose, kaolin, maltodextrin, malt extract, and microcrystalline cellulose. A non-limiting example of a commercially available colloidal hydrophilic silica is AEROSIL (Evonik). A stabilizer may also include a disaccharide, such as maltose, trehalose, lactose, sucrose, cellobiose, and any combination thereof. A stabilizer component may comprise from about 0.05% to about 10% by weight of a composition. For example, a stabilizer component may comprise from about 0.1% to about 5%, from about 0.1% to about 2%, or from about 0.1% to about 1% by weight of a composition.

### Antioxidants

The seed treatment component in some embodiments may comprise one or more oxidation control components, which may include one or more antioxidants (e.g., one or more of: ascorbic acid, ascorbyl palmitate, ascorbyl stearate, calcium ascorbate, carotenoids, lipoic acid, phenolic compounds (e.g., one or more flavonoids, flavones and/or flavonols), potassium ascorbate, sodium ascorbate, one or more thiols (e.g., glutathione, lipoic acid and/or N-acetyl cysteine), tocopherols, one or more tocotrienols, ubiquinone and/or uric acid) and/or one or more oxygen scavengers, such as ascorbic acid and/or sodium hydrogen carbonate.

### Polymers

The seed treatment component in some embodiments may comprise one or more agriculturally acceptable polymers, such as agar, alginate, carrageenan, cellulose, guar gum, locust bean gum, methylcellulose, pectin, polycaprolactone, polylactide, polyvinyl alcohol, polyvinyl pyrrolidone, sodium carboxymethyl cellulose, starch and/or xanthan gum. In an aspect, the one or more polymers is a natural polymer (e.g., agar, starch, alginate, pectin, cellulose, etc.), a synthetic polymer, a biodegradable polymer (e.g., polycaprolactone, polylactide, polyvinyl alcohol, etc.), or a combination thereof.

### Drying Agents

The seed treatment component in some embodiments may comprise one or more agriculturally acceptable drying agents, such as calcium stearate, one or more clays, graphite, magnesium stearate, magnesium sulfate, powdered milk, one or more silica powders, soy lecithin and/or talc.

### Anti-Freezing Agents

The seed treatment component in some embodiments may comprise one or more anti-freezing agents. for example, an anti-freezing agent may include one or more of ethylene glycol, alcohol, butanediol, pentanediol, mannitol, sorbitol, glycerol (glycerine), propylene glycol and/or urea. The antifreeze agent may be present in a composition at a concentration of at least 5 g/L, at least 10 g/L, at least 15 g/L, at least 20 g/L, at least 30 g/L, at least 40 g/L, at least 50 g/L, at least 60 g/L, at least 70 g/L, or at least 80 g/L, such as from about 1 to about 150 g/L, from about 10 to about 100 g/L, or from about 20 to about 80 g/L.

### Dendrimers

The seed treatment component in some embodiments may comprise one or more functionalized dendrimers to enhance the efficacy and/or stability of the composition. Non-limiting examples of classes of functionalized dendrimers include poly(amidoamine) (PAMAM, Generations 0-7), poly(amidoamine-organosilicone) (PAMAMOS), polypropylene imidine) (PPI, Generations 0-5), poly(benzylethers) (Frechet-type), Arobols (Newkome type), poly(phenylacetylenes) and surface engineered dendrimers (e.g. PEGylated dendrimers, glycodendrimers, peptide functionalized dendrimers, and galabiose-functionalized dendrimers). Dendrimer(s) may comprise at least 0.1% and up to 10% or more, or from about 1% to about 10%, of the composition by weight.

### Antifoam Agents

The seed treatment component in some embodiments may comprise one or more antifoam agents. Examples of antifoam agents include organosilicone or silicone-free compounds. Non-limiting examples of commercially available antifoam products include Break-Thru 0E441 (Evonik), Break-Thru AF9905 (Evonik), AGNIQUE DF 6889 (Cognis), AGNIQUE DFM 111S (Cognis), BYK-016 (BYK), FG-10 antifoam emulsion (Dow Corning), 1520-US (Dow Corning), 1510-US (Dow Corning), SAG 1538 (Momentive), and SAG 1572 (Momentive).

### Crystallization Inhibitors

The seed treatment component in some embodiments may comprise a crystallization inhibitor(s). Exemplary crystallization inhibitors include acrylic copolymers, polyethylene glycol, polyethylene glycol hydrogenated castor oil, and any combination thereof. The crystallization inhibitor may be present, for example, at a concentration from about 1% to about 10% by weight of the composition.

### Viscosity Modifying Agents

The seed treatment component in some embodiments may comprise one or more viscosity modifying agents. Examples of viscosity modifying agents include humic acid salts, fulvic acid salts, humin, and lignin salts, such as the sodium or potassium salt of humic acid.

The seed treatment component in some embodiments may comprise one or more additional excipients that improve the adhesion of the composition to a substrate or surface, such as a plant seed or other plant material, such as to provide a successful coating of the substrate or surface or otherwise impart improved characteristics to the adhesion or coating. Other substances may be added to the seed treatment component (e.g., coloring agents) to provide a visual indication of successful coating of the substrate or surface, such as the outer surface of a plant seed or other plant material.

### Film Coating Agents

In some embodiments, the seed treatment component comprises one or more film coating agents. Examples of such materials include, but are not limited to, albumins, alginates, celluloses, gums (e.g., cellulose gum, guar gum, gum arabic, gum combretum, xantham gum), methyl celluloses, nylons, pectins, polyacrylic acids, polycarbonates, polyethylene glycols (PEG), polyethylenimines (PEI), polylactides, polymethylacrylates (PMA), polyurethanes, polyvinyl alcohols (PVA), polyvinylpyrrolidones (PVP), propylene glycols, sodium carboxymethyl celluloses and starches.

Non-limiting examples of hygroscopic polymers that may be useful in the seed treatment component include AGRIMER™ polymers (e.g., 30, AL-10 LC, AL-22, AT/ATF, VA 3E, VA 31, VA 5E, VA 51, VA 6, VA 6E, VA 7E, VA 71, VEMA AN-216, VEMA AN-990, VEMA AN-1200, VEMA AN-1980, VEMA H-815MS; Ashland Specialty Ingredients, Wilmington, Del.), Atlox METASPERSE™(e.g., 550S; Croda International PLC, Edison, N.J.), EASYSPERSE™ polymers (Ashland Specialty Ingredients, Wilmington, Del.); DISCO™ AG polymers (e.g., L-250, L-280, L-285, L-286, L-320, L-323, L-517, L-519, L-520, L800; Incotec Inc., Salinas, Calif.), KELZAN® polymers (Bri-Chem Supply Ltd., Calgary, Alberta, Calif.), SEEDWORX™ polymers (e.g., Bio 200; Aginnovation, LLC, Walnut Groove, Calif.), TABULOSE® gels (e.g., SC-580, SC-612, SC-613, SC-681; Blanver Farmoquimica, Boca Raton, Fla.), TICAXAN® xanthan powders (TIC Gums, White Marsh, Md.) and combinations thereof.

Film-forming polymers may be present in any suitable amount(s)/concentration(s). In some embodiments, the film-forming polymer(s) comprise(s) about 1 to about 75% (by weight) of the seed treatment component. In some embodiments, the film-forming polymer(s) comprise(s) about 5 to about 6, 7, 8, 9, 10, 11, 12, 13, 14, 15, 16, 17, 18, 19, 20, 21, 22, 23, 24, 25, 30, 35, 40, 45 or 50% (by weight) of the composition. In some embodiments, the film-forming polymer amount/concentration is about 5 to about 15% (by weight) of the seed treatment component. In some embodiments, the film-forming polymer amount/concentration is about 10 to about 25% (by weight) of the seed treatment component.

### Colorants

In some embodiments, the seed treatment component comprises one or more colorants. Examples of such materials include, but are not limited to, organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, and xanthene.

### Dispersants

In some embodiments, the seed treatment component comprises one or more dispersants, including, but not limited to, surfactants and wetting agents. Dispersants may be used to maintain a homogeneous or even distribution of particles or cells in a suspension, such as an even or homogeneous distribution of a microbial strain or isolate, which may be particularly useful for solid or dried formulations of a microbe and/or liquid formulations or fermentates. In addition to maintaining an even distribution of the microbe in a final composition or formulation and during application of a composition or formulation to a plant, plant part or plant seed, a dispersant or wetting agent may also facilitate mixing of a microbe with other ingredients and solvents of a microbial formulation or composition and avoid aggregation or clumping of particles, or their adherence to container walls, etc., during formulation of a microbial composition. The seed treatment component may comprise a primary dispersant in combination with one or more secondary dispersants, and the primary and secondary dispersants may be different types (e.g., non-ionic, cationic, and/or anionic). Wetting agents may be used with compositions applied to soils, particularly hydrophobic soils, to improve the infiltration and/or penetration of water into a soil. The wetting agent or dispersant may be an adjuvant, oil, surfactant, buffer, acidifier, or combination thereof. The wetting agent or dispersant may be a surfactant, such as one or more non-ionic surfactants, one or more cationic surfactants, one or more anionic surfactants, or any combination thereof

The seed treatment component in some embodiments may comprise at least 5 g/L, at least 10 g/L, at least 15 g/L, at least 20 g/L, at least 25 g/L, at least 30 g/L, at least 35 g/L, at least 40 g/L, at least 45 g/L, or at least 50 g/L of a dispersant(s). In some embodiments, the dispersant may be from about 1 to about 100 g/L, from about 5 to about 75 g/L, or from about 20 to about 50 g/L. The amount of dispersants may also be expressed as a percentage by weight of a composition, such as about 0. 5% to about 20%, from about 0. 5% to about 10%, from about 0. 5% to about 5%, from about 0. 5% to about 8%, from about 0. 5% to about 5%, or from about 1% to about 4% by weight of the composition.

### Surfactants

In some embodiments, seed treatment component comprises one or more anionic surfactants. for example, the seed treatment component may comprise one or more water-soluble anionic surfactants and/or one or more water-insoluble anionic surfactants. In some embodiments, the seed treatment component of the present disclosure may comprise one or more anionic surfactants chosen from acyl isethionates, acyl sarconsinates, alcohol ether carboxylates, alcohol ether sulfates, alcohol sulfates, alkyl amide sulfonates, alkylamide sulfosuccinates, alkyl amido ether sulfates, alkyl aryl ether sulfates, alkyl aryl polyether sulfates, alkyl aryl sulfates, alkyl aryl sulfonates, alkyl benzene sulfonates, alkyl carboxylates, alkyl carboxylates, alkyl diphenyloxide sulfonate, alkyl ether phosphates, alkyl ether sulfates, alkyl ether sulfosuccinates, alkyl naphthalene sulfonates, alkyl phosphates, alkyl sulfates, alkyl sulfoacetates, alkyl sulfonates, alkyl sulfosuccinamate, alkyl sulfosuccinamates, alkyl sulfosuccinates, alpha-olefin sulfonates, aryl sulfonates, benzene sulfonates, cumene sulfonates, dioctyl sodium sulfosuccinate, ethoxylated sulfosuccinates, lignin sulfonates, linear alkylbenzene sulfonates, mono- or diphosphate esters of polyalkoxylated alkyl alcohols or alkyl phenols, mono- or disulfosuccinate esters of alcohols or polyalkoxylated alkanols, monoglyceride sulfates, N-acyl-N-alkyltaurates, N-acyl taurates, paraffin sulfonates, perfluorobutanesulfonate, perfluorooctanesulfonate, phenol ether carboxylates, phosphate ester, styrene acrylic polymers, sulfosuccinates, toluene sulfonates, xylene sulfonates.

Other non-limiting examples of commercially available anionic surfactants include sodium dodecyl sulfate (Na-DS, SDS), MORWET D-425 (a sodium salt of alkyl naphthalene sulfonate condensate, available from Akzo Nobel), MORWET D-500 (a sodium salt of alkyl naphthalene sulfonate condensate with a block copolymer, available from Akzo Nobel), sodium dodecylbenzene sulfonic acid (Na-DBSA) (Aldrich), diphenyloxide disulfonate, naphthalene formaldehyde condensate, DOWFAX (Dow), dihexylsulfosuccinate, and dioctylsulfosuccinate, TWEEN®, alkyl naphthalene sulfonate condensates, and salts thereof.

In some embodiments, seed treatment component comprises one or more cationic surfactants. Non-limiting examples of cationic surfactants include mono alkyl quaternary amine, fatty acid amide surfactants, amidoamine, imidazoline, and polymeric cationic surfactants. for example, in some embodiments, the seed treatment component comprises one or more pH-dependent amines and/or one or more quaternary ammonium cations, optionally one or more cationic surfactants chosen from alkyltrimethylammonium salts (e.g., cetyl trimethylammonium bromide, cetyl trimethylammonium chloride), cetylpyridinium chloride, benzalkonium chloride, benzethonium chloride, 5-Bromo-5-nitro-1,3-dioxane, dimethyldioctadecylammonium chloride, cetrimonium bromide, dioctadecyldimethylammonium bromide and/or octenidine dihydrochloride.

In some embodiments, the seed treatment component comprises one or more non-ionic surfactants. for example, the seed treatment component may comprise one or more water-soluble non-ionic surfactants and/or one or more water-insoluble non-ionic surfactants, optionally one or more non-ionic surfactants chosen from alcohol ethoxylates (e.g., TERGITOL™ 15-S surfactants, such as TERGITOL™ 15-S-9 (The Dow Chemical Company, Midland, mich.)), alkanolamides, alkanolamine condensates, carboxylic acid esters, cetostearyl alcohol, cetyl alcohol, cocamide DEA, dodecyldimethylamine oxides, ethanolamides, ethoxylates of glycerol ester and glycol esters, ethylene oxide polymers, ethylene oxide-propylene oxide copolymers, glucoside alkyl ethers, glycerol alkyl ethers (e.g.,), glycerol esters, glycol alkyl ethers (e.g., polyoxyethylene glycol alkyl ethers, polyoxypropylene glycol alkyl ethers,), glycol alkylphenol ethers (e.g., polyoxyethylene glycol alkylphenol ethers,), glycol esters, monolaurin, pentaethylene glycol monododecyl ethers, poloxamer, polyamines, polyglycerol polyricinoleate, polysorbate, polyoxyethylenated fatty acids, polyoxyethylenated mercaptans, polyoxyethylenated polyoxyproylene glycols, polyoxyethylene glycol sorbitan alkyl esters, polyethylene glycol-polypropylene glycol copolymers, polyoxyethylene glycol octylphenol ethers, polyvinyl pyrrolidones, sugar-based alkyl polyglycosides, sulfoanylamides, sorbitan fatty acid alcohol ethoxylates, sorbitan fatty acid ester ethoxylates, sorbitan fatty acid ester and/or tertiary acetylenic glycols.

Examples of non-ionic surfactants include sorbitan esters, ethoxylated sorbitan esters, alkoxylated alkylphenols, alkoxylated alcohols, block copolymer ethers, and lanolin derivatives. In accordance with an aspect, the surfactant comprises an alkylether block copolymer. Other non-limiting examples of water insoluble non-ionic surfactants include alkyl and aryl glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, or combinations thereof. Also included are EO/PO block copolymers (EO is ethylene oxide, PO is propylene oxide), EO polymers and copolymers, polyamines, and polyvinyl pyrrolidones.

Further non-limiting examples of water soluble non-ionic surfactants include sorbitan fatty acid alcohol ethoxylates and sorbitan fatty acid ester ethoxylates. further non-limiting examples of commercially available non-ionic surfactants include SPAN 20, SPAN 40, SPAN 80, SPAN 65, and SPAN 85 (Aldrich); TWEEN 20, TWEEN 40, TWEEN 60, TWEEN 80, and TWEEN 85 (Aldrich); IGEPAL CA-210, IGEPAL CA-520, IGEPAL CA-720, IGEPAL CO-210, IGEPAL CO-520, IGEPAL CO-630, IGEPAL CO-720, IGEPAL CO-890, and IGEPAL DM-970 (available from Aldrich); Triton X-100 (Aldrich); BRIJ S10, BRIJ S20, BRIJ 30, BRIJ 52, BRIJ 56, BRU 58, BRU 72, BRU 76, BRIJ 78, BRIJ 92V, BRU 97, and BRU 98 (Aldrich); PLURONIC L-31, PLURONIC L-35, PLURONIC L-61, PLURONIC L-81, PLURONIC L-64, PLURONIC L-121, PLURONIC 10R5, PLURONIC 17R4, and PLURONIC 31R1 (Aldrich); Atlas G-5000 and Atlas G-5002L (Croda); ATLOX 4912 and ATLOX 4912-SF (Croda); and SOLUPLUS (BASF), LANEXOL AWS (Croda). The seed treatment component may comprise at least one or more non-ionic surfactants, such as at least one water-insoluble non-ionic surfactant, at least one water soluble non-ionic surfactant, or combinations thereof. In still another aspect, the seed treatment component comprises a combination of non-ionic surfactants having hydrocarbon chains of substantially the same length.

In some embodiments, the seed treatment component comprises one or more zwitterionic surfactants. for example, the seed treatment component may comprise one or more betaines and/or one or more sultaines, optionally one or more zwitterionic surfactants chosen from 3-[(3-cholamidopropyl)dimethylammonio]-1-propanesulfonate, cocamidopropyl betaine, cocamidopropyl hydroxysultaine, phosphatidylserine, phosphatidylethanolamine, phosphatidylcholine and/or one or more sphingomyelins.

In some embodiments, the seed treatment component comprises one or more ionic surfactants (e.g., one or more ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, sorbitan fatty acid alcohol ethoxylates and/or sorbitan fatty acid ester ethoxylates).

In some embodiments, the seed treatment component may comprise one or more soaps and/or organosilicone surfactants. for example, in some embodiments, the seed treatment component comprises one or more alkali metal salts of fatty acids.

### Wetting Agents

In some embodiments, the seed treatment component comprises one or more wetting agents. In certain embodiments, the wetting agent is chosen from an adjuvant, oil, surfactant, buffer, and acidifier. For example, in some embodiments, the seed treatment component comprises one or more naphthalene sulfonates, optionally one or more alkyl naphthalene sulfonates (e.g., sodium alkyl naphthalene sulfonate), one or more isopropyl naphthalene sulfonates (e.g., sodium isopropyl naphthalene sulfonate) and/or one or more butyl naphthalene sulfonates (e.g., sodium n-butyl naphthalene sulfonate).

### pH

According to some embodiments, the method comprises obtaining an agricultural compositions that has a desired pH in a range from about 4.5 to about 9. 5. For example, agricultural compositions may have a pH in a range from about 6 to about 8, or a pH of about 5, 5.5, 6, 6.5, 7, 7.5, 8 or 8.5. To maintain a desired pH, an agricultural composition in some embodiments may comprise a buffer solution. Buffers may be selected to provide an aqueous composition having a pH of less than 10, typically from about 5 to about 9, from about 6 to about 8, or about 7. Buffer solutions suitable for a variety of pH ranges are known in the art.

### Microbes Detected

In certain embodiments, the methods described herein include obtaining an agricultural composition which comprises more than one microbe to be detected.

In certain embodiments, the methods described herein include obtaining an agricultural composition which comprises a microbial community. The term “microbial community,” as used herein, refers to one or more populations of microbes found in a shared environment. For example, a shared environment can be an agricultural composition.

In certain embodiments, the methods described herein include obtaining an agricultural composition which comprises bacteria, fungi, and/or archaea to be detected.

### Bacteria

In certain embodiments, the method described herein comprises detecting one or more than one bacteria from an agricultural composition. The terms “bacteria” and “bacterium” refer to prokaryotic organisms, including those within the phyla in the Kingdom Prokaryote. All forms of bacteria are included within this definition including cocci, bacilli, spirochetes, spheroplasts, protoplasts, etc.

In some embodiments, the method described herein comprises detecting a bacterial endophyte or a root or phylloplane colonizer from an agricultural composition. In certain embodiments, the bacteria to be detected in the methods disclosed herein may exist in vegetative form, spore form, and combinations thereof. In some embodiments, at least 1% of the bacteria present comprise spores. In certain embodiments, at least 5%, at least 10%, at least 20%, at least 30%, at least 40%, at least 50%, at least 75%, at least 80%, at least 90%, or at least 95% or more of the bacteria to be detected comprise spores.

In some embodiments, the method described herein comprises detecting a bacterial aggregate. Bacterial aggregates are composed of bacteria embedded in an organic gelatinous structure composed of one or more matrix polymers that are secreted by the resident microbes. In some embodiments, the method described herein comprises detecting a biofilm or an aggregation of bacteria surrounded by an extracellular matrix, slime or matrix on a surface, or slime adherent on a surface.

In some embodiments, the method described herein comprises detecting bacteria that is viable but nonculturable. Viable but nonculturable (VBNC) bacteria refers to bacteria that are in a state of low metabolic activity and do not divide, but are alive and can become culturable once resuscitated.

In some embodiments, the method described herein comprises detecting Gram-negative bacteria. In some embodiments, the method described herein comprises detecting Gram-positive bacteria. “Gram negative” and “Gram positive” refer to staining patterns with the Gram-staining process and well known in the art.

### Fungi

In certain embodiments, the method described herein comprises detecting fungi in an agricultural composition. As used herein, the term “fungi” refers to eukaryotic organisms such as the molds and yeasts, including dimorphic fungi.

### Growth Phase

In certain embodiments, the method described herein comprises detecting a viable microbe in an agricultural composition that is in a mid-logarithmic growth phase. The logarithmic phase of growth is a pattern of balanced growth wherein the cells are dividing regularly by binary fission, and are growing by geometric progression. The cells divide at a constant rate depending upon the composition of the growth medium and the conditions of incubation.

In certain embodiments, the method described herein comprises detecting a viable microbe in an agricultural composition that is in a stationary growth phase. In the stationary phase, the number of new cells produced balances the number of cells that die, resulting in a steady state. In some embodiments, the microbe's nutrients in the agricultural composition are limited and/or metabolic products have accumulated to such a level that they inhibit cell growth in the stationary growth phase. Cells capable of making an endospore will activate the necessary genes during this stage, to initiate the sporulation process.

### Detection

In the methods disclosed herein, the method comprises detecting the amount of the pre-rRNA of the microbe to be detected in an agricultural composition. In the methods disclosed herein, pre-rRNA can be detected or measured by a variety of methods including an amplification assay, a hybridization assay, a sequencing assay, or an array. Non-limiting examples of methods to detect pre-rRNA by amplification assay, hybridization assay, sequencing assay, or array include reverse-transcription quantitative polymerase chain reaction (RT-qPCR) such as TaqMan®; end-point TaqMan® RT-qPCR on conventional and digital platforms, e.g. Formulatrix dPCR and BioRad digital droplet RT-qPCR (ddPCR); Northern blotting; in situ hybridization assays; microarray analysis multiplexed hybridization-based assays, e.g., QuantiGene 2.0 Multiplex Assay from ThermoFisher and nCounter Analysis System by NanoString Technologies; serial analysis of gene expression (SAGE); cDNA-mediated annealing, selection, extension, and ligation; nucleic acid immunoassay, direct sequencing, sequencing by synthesis, or pyrosequencing; targeted and conventional RNA-sequencing; massively parallel sequencing; next generation sequencing; high performance liquid chromatography (HPLC) fragment analysis; capillarity electrophoresis; mass spectrometry, including SELDI, MALDI; and other known methods.

In some embodiments, the method comprises detecting pre-rRNA of the microbe to be detected in the agricultural composition by immobilizing the pre-rRNA on a solid surface and contacting the pre-rRNA with a probe, e.g., in a microarray, dot blot or Northern format. A skilled artisan can readily adapt known RNA detection methods for use in detecting the pre-rRNA.

In some embodiments, the method comprises detecting pre-rRNA of the microbe to be detected in the agricultural composition by amplification reactions and/or reactions in which probes are linked to a solid support and used to quantify RNA may be used. Alternatively, in some embodiments, the method comprises detecting pre-rRNA of the microbe to be detected in the agricultural composition by linking the RNA, or DNA copy of the RNA, to a solid support and quantifying using a probe to the sequence of interest.

In some embodiments, the method comprises detecting pre-rRNA of the microbe to be detected in the agricultural composition by first reverse transcribing the pre-RNA and quantifying the resulting cDNA. In some embodiments, the method comprises detecting pre-rRNA of the microbe to be detected in the agricultural composition by using RT-PCR or other quantitative amplification techniques known in the art. Alternative methods for determining the level of pre-rRNA in a sample of the agricultural composition may involve other nucleic acid amplification methods such as ligase chain reaction, self-sustained sequence replication, transcriptional amplification system , rolling circle replication or any other nucleic acid amplification method well known to those of skill in the art.

### Microarrays

In some embodiments, the method comprises detecting pre-rRNA of the microbe to be detected in the agricultural composition using microarrays. Microarrays provide one method for the simultaneous measurement of the expression levels of large numbers of pre-rRNA. Each array consists of a reproducible pattern of capture probes attached to a solid support. Labeled RNA or DNA is hybridized to complementary probes on the array and detected by laser scanning. Hybridization intensities for each probe on the array are determined and converted to a quantitative value representing relative expression levels. High-density oligonucleotide arrays are particularly useful for determining the expression profile for a large number of RNA's in a sample.

Techniques for the synthesis of microarrays using mechanical synthesis methods are known in the art. Although a planar array surface is often employed the array may be fabricated on a surface of virtually any shape or a multiplicity of surfaces. Arrays may be peptides or nucleic acids on beads, gels, polymeric surfaces, fibers such as fiber optics, glass or any other appropriate substrate known in the art.

In some embodiments, gene-specific probes and/or primers are used in hybridization assays to detect pre-rRNA expression. The probes and/or primers may be labeled with any detectable moiety or compound, such as a radioisotope, fluorophore, chemiluminescent agent, and enzyme.

Probes and primers for use in the methods disclosed herein to detect the pre-rRNA can be selected using known algorithms that utilize binding energies, base composition, sequence complexity, cross-hybridization binding energies, and secondary structure.

The probes and primers necessary for practicing the methods for detecting the pre-rRNA of the microbe in the agricultural composition can be synthesized and labeled using well known techniques. Oligonucleotides used as probes and primers may be chemically synthesized per the solid phase phosphoramidite triester method.

In some embodiments, probes used to detect the pre-rRNA of the microbe in the methods disclosed herein can be obtained, e.g., by polymerase chain reaction (PCR) amplification of genomic DNA or RNA or cloned sequences. PCR primers are selected based on a known sequence of the genome that will result in amplification of specific fragments of genomic DNA. Computer programs that are well known in the art are useful in the design of primers with the required specificity and optimal amplification properties. In some embodiments, the probe is between 10 bases and 50,000 bases, usually between 300 bases and 1,000 bases in length. It will be apparent to one skilled in the art that controlled robotic systems are useful for isolating and amplifying nucleic acids. In some embodiments, in situ hybridization is employed to assess pre-rRNA levels.

### RT-qPCR

In certain embodiments, the method comprises detecting the amount of the at least one pre-rRNA from at least one microbe via RT-qPCR.

In certain embodiments, RT-qPCR may be used to quantify species-specific pre-rRNA from a sample to determine the pre-rRNA stimulation values. The species-specific DNA may be amplified from pre-rRNA-containing samples using a reverse transcription-polymerase chain reaction. The amplification step uses a first primer complementary to the microbe's pre-rRNA region, a second primer complementary to the microbe's mature rRNA, and performing multiple cycles of amplification using the first primer and the second primer yields detectable levels of amplified species-specific DNA.

In some embodiments, the method comprises quantifying species-specific DNA by using a fluorescently labeled hybridizing probe complementary to the microbe's mature rRNA, wherein a first primer is complementary to a microbe's pre-rRNA region and a second primer complementary to the microbe's mature rRNA, and performing multiple cycles of amplification using the fluorescently labeled hybridizing probe to generate a quantifiable fluorescence signal. The quantifiable fluorescence signal compared to a standard curve constructed from known concentrations provides an absolute quantification of the microbe's pre-rRNA; quantifiable fluorescence signal compared to an internal reference gene provides a relative quantification.

### Timing and Storage

Maintaining the viability of microbial inoculants is a challenge in development of new products. The methods disclosed herein may be used to evaluate the shelf life of microbial inoculants. In some embodiments, the method includes detecting pre-rRNA of the microbe to be detected in the agricultural composition over a period of time and different storage conditions.

In certain embodiments, the method comprises detecting pre-rRNA of the microbe to be detected in the agricultural composition after treatment on a seed. In certain embodiments, the method comprises detecting pre-rRNA of the microbe to be detected in the agricultural composition after a period of 3 days. In certain embodiments, the method comprises detecting pre-rRNA of the microbe to be detected in the agricultural composition after a period of one year. In certain embodiments, the method comprises detecting pre-rRNA of the microbe to be detected in the agricultural composition after a period of two years. In certain embodiments, the method comprises detecting pre-rRNA of the microbe to be detected in the agricultural composition after a period of three years.

In certain embodiments, the method includes detecting pre-rRNA of the microbe to be detected in the agricultural composition at different storage conditions. Non-limiting examples of storage conditions include storing the agricultural composition including the microbe to be detected at temperature between about 4° and about 40° C. In certain embodiments, the agricultural composition including the microbe to be detected is stored at a relative humidity between about 5% and about 80%.

### EXAMPLES

Non-limiting examples of detecting microbes, more specifically to methods for detecting viable microbes in an agricultural composition are provided.

### Detecting Microbes on Treated and Untreated Seed

### Microbe Strains and Growth Conditions

Cultures were grown in Lysogeny Broth (LB), at 28° C. P. entomophila and B. thuringiensis cells were grown and harvested at either mid-log or late-log phase. The cells were washed with TBS buffer, followed by a 30% glycerol solution. These cells were frozen and kept at −80° C. P. restrictum cells were grown overnight and harvested and sub-cultured into fresh medium. The sub-culture was harvested after 4 hours of growth. The cells were washed with TBS buffer, followed by a 30% glycerol solution. These cells were frozen and kept at −80° C.

### Artificial Microbial Communities

Two microbial communities were made by combining strains from culture collection. All isolates started from glycerol stocks at a concentration of 10⁸ cells per milliliter. Mix 1 was made to a final volume of 400 μL with approximately 10⁷ cells of a target P. entomophila strain, 10⁷ cells of a Rhizobium sp. strain, 5⁶ cells of a Pseudomonas sp. strain, 5⁶ cells of a Luteibacter sp. strain, and 10⁷ cells of a Flavobacterium sp. strain. Mix 2 was made to a final volume of 400 μL with approximately 10⁷ cells of a target B. thuringiensis strain, 10⁷ cells of a Herbiconiux solani strain, 10⁷ cells of a Bacillus sp. strain, and 10⁷ cells of a Rhizobium grahamii strain.

### Inactivation

Cultures were heat-killed by incubating at 80° C. for 30 minutes.

### Seed Treatment and Inoculation

Soybean seeds were pretreated with chemical commercially available seed treatment fungicides, and red colorant in a batch seed treater prior to application of inoculant. Treated seed was placed into a bag, inoculant applied, and the bag closed tightly to create an air pocket for the seed to be swirled vigorously for ˜1 min to evenly coat the individual seed. Liquid microbial inoculant was applied to each seed batch to provide an estimated 1×10⁶ cells per seed. The inoculated seeds were then incubated 0-7 days prior to microbe extraction.

### Microbe Extraction

For each sample, 10 inoculated seeds were suspended in 10 mL TBS buffer and vortexed for 4 minutes.

Nutritional Stimulation

Cultures and mixed communities were incubated in Lysogeny Broth (LB), at 30° C. for 30 minutes, except in the case of a P. bilaii strain, wherein the strain was stimulated in a time course for 30 min, 3 hours, and 24 hours in Lysogeny Broth (LB).

Pre-rRNA Isolation

Immediately following stimulation, cells were pelleted by centrifugation at 4° C. at 10,000× g for 5 min. Supernatant was discarded and pellets suspended in Tri Reagent (Sigma) with or without Max Bacterial RNA Reagent (Life Technologies). The suspended cells were transferred to a lysis tube containing a 0.1 mm silica matrix or 2 ball bearings and bead beaten for 4 minutes. Total RNA was purified from lysed cells using the commercially available Direct-zol RNA kit with DNase treatment (Zymo Research).

### RT-qPCR

Total RNA was normalized by mass determined spectrophotometrically by Nanodrop and cDNA synthesized by reverse transcription (High Capacity cDNA Reverse Transcriptase kit by Life Technologies) using random primers. 16S pre-rRNA (prokaryotes) or ITS1 (fungal) was detected by RT-qPCR using a Taqman assay. Primers and a probe were developed specifically to detect pre-rRNA (prokaryotes) or ITS1 (fungal) for each species. Reactions were set up using Quanta PerfeCTa Fastmix II master mix and cycled on an automated CFX96 (Bio-Rad) as follows: 95° C. for 2 minutes, 40 cycles of 95° C. for 10 seconds and 60° C. for 30 seconds. Results were reported as threshold cycle (Ct), the amplification cycle at which the fluorescence of the sample is above background noise. Expression (also referred to as Relative Quantity) in arbitrary units was calculated in some instances which is a result of this equation: 2\hat{ }-(Ct -35).

### Results and Discussion

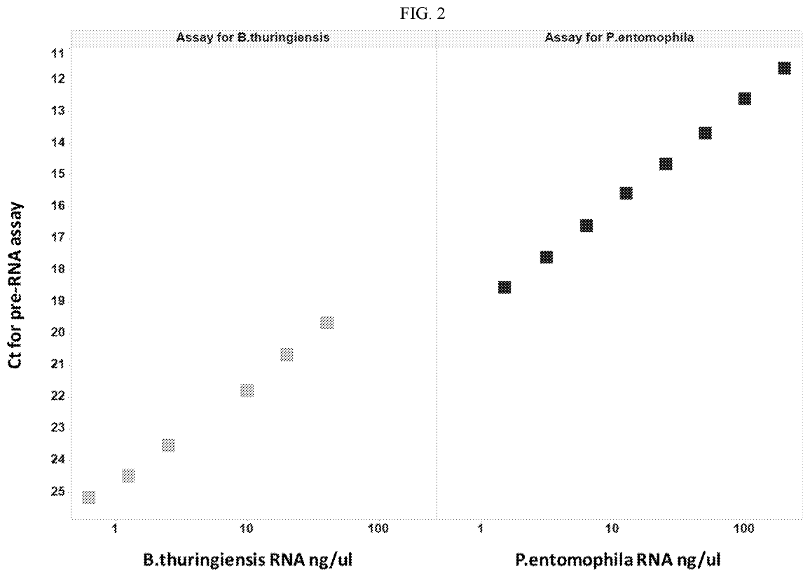

A linear response is observed for RT-qPCR assays based on dilution series of total RNA measured by nanodrop, as shown in FIG. 2.

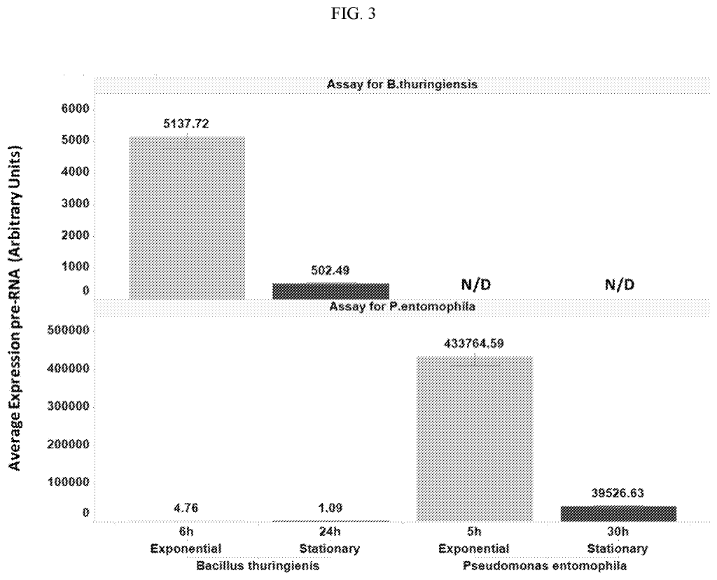

The amount of pre-rRNA present in the samples grew to exponential verses stationary phase for Pseudomonas entomophila and Bacillus thuringienis, respectively. A 10-fold difference was observed between exponential and stationary phase growth, as shown in FIG. 3.

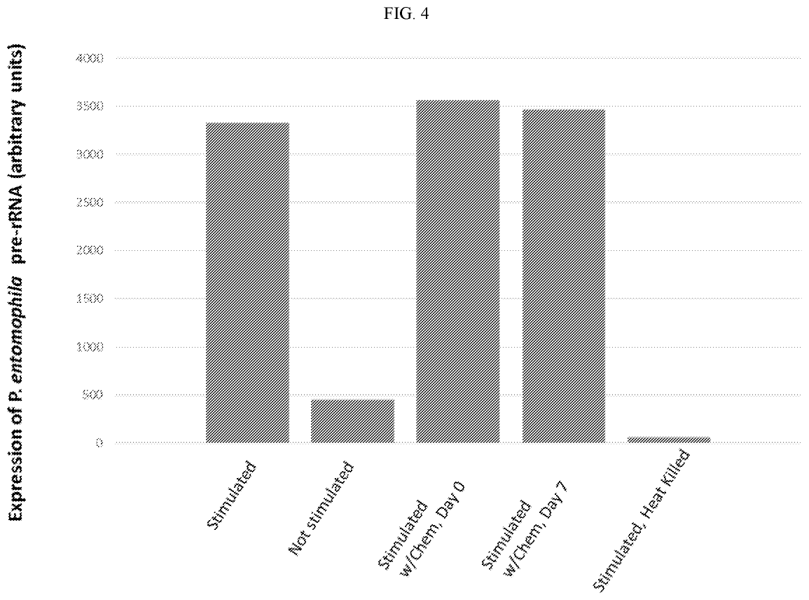

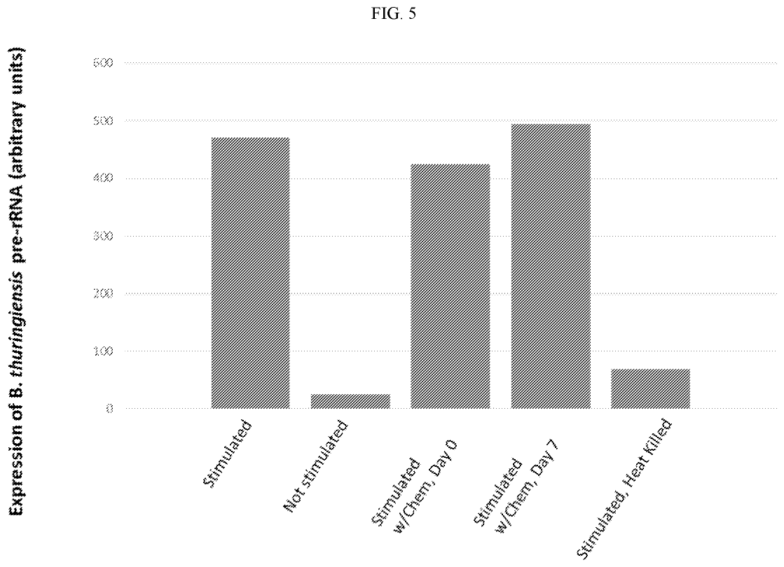

The results showed pre-rRNA signal can be measured with seed-treated Pseudomonas entomophila and Bacillus thuringiensis in the presence of seed treatment commercially available fungicide chemistries. The treated seeds were incubated at room temperature for 0 and 7 days. Cells were washed off the seed and nutritionally stimulated for 30 minutes before measured pre-rRNA. The water incubated, and heat-killed cells (incubated with LB) did not induce pre-rRNA as expected, while nutritionally stimulated cells produced a strong signal, as shown in FIG. 4 and FIG. 5.

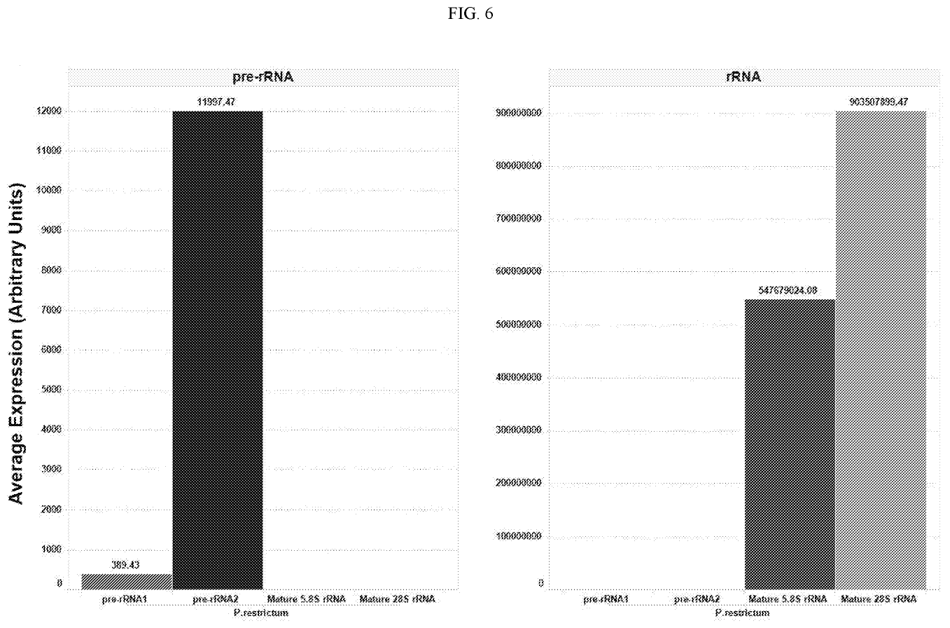

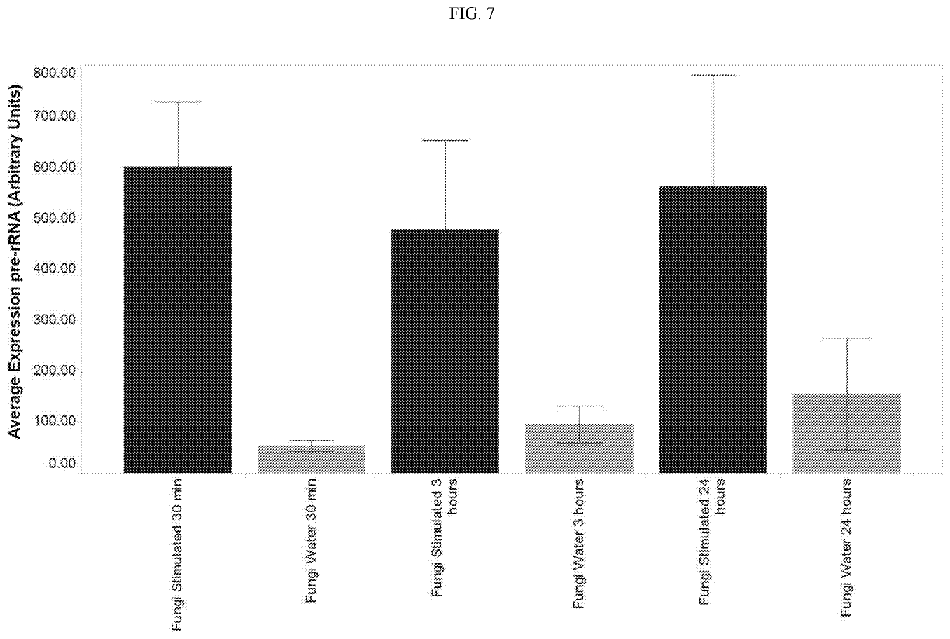

Both mature and pre-rRNA was detected from the fungal species Penicillium restrictum (as shown in FIG. 6) and Penicillium bilaii (as shown in FIG. 7). Thus, the method may be used to measure viability in both eukaryotes and prokaryotes.

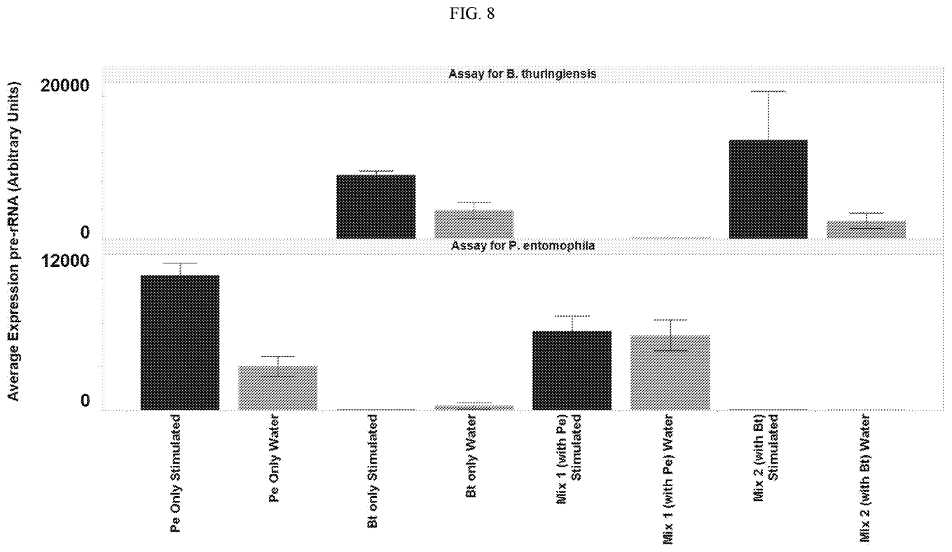

Both mature and pre-rRNA were detected in artificial microbial communities, as shown in FIG. 8. P. entomophila and B. thuringiensis were detected in artificial communities containing additional species: Rhizobium sp., Pseudomonas sp., Luteibacter sp., and Flavobacterium sp. P. entomophila and B. thuringiensis relative to rRNA amounts. Signal from assays specific to the target organisms were below the limit of detection when target was not present in mixed microbial community. This demonstrates the strain specificity of the pre-rRNA detection.

The disclosed viability assay is sensitive, specific, and detects microbes in the presence of seed treatment components, including the commercially available seed treatment fungicides. Viability cannot be detected or stimulated in heat-killed microbes, thus distinguishing viable microbes from non-viable microbes.

Viable but nonculturable (VBNC) cells were detected using this assay, when comparing plates to stimulated cells. Observing a pre-rRNA signal established viability for cells that had no colonies when plated in a conventional detection method. Since VBNC cells are metabolically active but cannot replicate other methods of measuring viability which rely on cell replication, incorrectly assess non-viability when the cells are viable but in VBNC state.

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A method for detecting a viable microbe in an agricultural composition, the method comprising:
a. obtaining a first sample of an agricultural composition;
b. obtaining a control sample of the agricultural composition;
c. nutritionally stimulating the first sample;
d. incubating the first sample;
e. detecting an amount of at least one pre-rRNA from at least one microbe in the first sample;
f. detecting an amount of at least one pre-rRNA from at least one microbe in the control sample and
g. comparing the amount of the at least one pre-rRNA from the at least one microbe in the first sample to the amount of the at least one pre-rRNA from the at least one microbe in the control sample; wherein a greater amount of detected pre-rRNA in the first sample than in the control sample indicates the presence of a viable microbe.

2. The method of claim 1, wherein the agricultural composition comprises an agriculturally acceptable carrier, plant material, a seed, soil, more than one microbe, or a microbial community.

3-6. (canceled)

7. The method of claim 1, wherein the agricultural composition comprises bacteria, fungi, and/or archaea.

8. (canceled)

9. The method of claim 7, wherein a) the bacteria in the agricultural composition comprises bacterial aggregates, a Gram-negative bacteria, or a Gram-positive bacteria; or b) the bacteria is viable but nonculturable.

10-13. (canceled)

14. The method of claim 1, wherein the viable microbe in the agricultural composition is in a mid-logarithmic growth phase or a stationary growth phase.

15. (canceled)

16. The method of claim 1, wherein detecting the amount of the at least one pre-rRNA from at least one microbe in the control sample and detecting the amount of the at least one pre-rRNA from at least one microbe in the first sample is determined via RT-qPCR.

17. (canceled)

18. The method of claim 1, wherein the agricultural composition comprises a seed treatment component.

19. (canceled)

20. The method of claim 18, wherein the seed treatment component comprises:
a) a pesticide;
b) a microbial inoculant;
c) one or more agriculturally acceptable nutrients and/or fertilizers;
d) one or more plant signal molecules; or
e) one or more adherents, adhesives binders, buffers, coating agents, colorants, dispersants, fillers, polymers, polysaccharides, surfactants, and/or wetting agents.

21. The method of claim 20, wherein the pesticide is selected from the group consisting of at least one or more biocides, fungicides, herbicides, insecticides, miticides, nematicides, rodenticides, tioxazafen, clothianidin, ipconazole, imidacloprid, prothiconazol, fluoxastrobin, metalaxyl, trifloxystrobin, metalaxyl, and combinations thereof.

22-25. (canceled)

26. A method for determining the viability of a microbial inoculant, the method comprising:
a. inoculating an agricultural composition with a microbe inoculant;
b. after a period of time, obtaining a first sample and a control sample from the agricultural composition with the microbe inoculant;
c. nutritionally stimulating the first sample;
d. incubating the first sample;
e. detecting the amount of the at least one pre-rRNA from at least one microbe in the first sample;
f. detecting the amount of the at least one pre-rRNA from at least one microbe in the control sample;
g. comparing the amount of the at least one pre-rRNA from at least one microbe in the first sample to the amount of the at least one pre-rRNA from at least one microbe in the control sample; and
h. quantifying viability of the at least one microbe in the first sample based on comparing the amount of the at least one pre-rRNA from at least one microbe in the first sample to the amount of the at least one pre-rRNA from at least one microbe in the control sample.

27. The method of claim 26, wherein the agricultural composition comprises an agriculturally acceptable carrier, plant material, or a seed.

28. (canceled)

29. The method of claim 26, wherein the first sample and control sample comprise:
a) more than one microbe; or
b) a microbial community.

30. (canceled)

31. The method of claim 26, wherein the at least one microbe is selected from the group consisting of bacteria, fungi, and archaea.

32. The method of claim 31, wherein the at least one microbe comprises bacteria.

33. The method of claim 32, wherein a) the at least one microbe comprises bacterial aggregates, a Gram-negative bacteria, or a Gram-positive bacteria; or b) the bacteria is viable but nonculturable.

34-37. (canceled)

38. The method of claim 26, wherein
a) the period of time is at least 3 days, at least one year, at least two years or at least three years or;
b) the at least one microbe in the agricultural composition is in a mid-logarithmic growth phase or a stationary growth phase.

39-43. (canceled)

44. The method of claim 26, wherein detecting the amount of the at least one pre-rRNA from at least one microbe in the control sample and first sample is determined via RT-qPCR.

45. (canceled)

46. The method of claim 45, wherein the agricultural composition comprises a seed treatment component.

47. The method of claim 46, wherein the seed treatment component comprises:
a) a pesticide;
b) one or more agriculturally acceptable nutrients and/or fertilizers;
c) one or more plant signal molecules; or
d) one or more adherents, adhesives, binders, buffers, coating agents, colorants, dispersants, fillers, polymers, polysaccharides, surfactants, and/or wetting agents.

48. The method of claim 47, wherein the pesticide is selected from the group consisting of at least one or more biocides, fungicides, herbicides, insecticides, miticides, nematicides, rodenticides, tioxazafen, clothianidin, ipconazole, imidacloprid, prothiconazol, fluoxastrobin, metalaxyl, trifloxystrobin, metalaxyl, and combinations thereof.

49-52. (canceled)
